(19) United States
(12) Patent Application Publication
(10) Pub. No.: US 2023/0383535 A1
(43) Pub. Date: Nov. 30, 2023

(54) MODULAR WALL COMPONENT AND A METHOD FOR MANUFACTURING A MODULAR WALL COMPONENT

(71) Applicant: Green Canopy NODE Technologies, INC, Seattle, WA (US)

(72) Inventors: Darrin Russell Griechen, Spokane, WA (US); Eliot Thomas Siegel, Seattle, WA (US); Rosalie Elena Schlenk, Seattle, WA (US); Abigail Christine Willets, New Orleans, LA (US); Tulio Caesar Squarcio, Seattle, WA (US); Anthony Carmine Prato, Holland, PA (US)

(21) Appl. No.: 18/318,108

(22) Filed: May 16, 2023

Related U.S. Application Data
(60) Provisional application No. 63/346,101, filed on May 26, 2022.

(51) Int. Cl. E04B 2/72; E04B 2/74

(52) U.S. Cl. CPC E04B 2/721; E04B 2/74; E04B 2002/7488; E04B 2002/7461

(57) ABSTRACT

A modular wall component is provided. One aspect of the modular wall component includes at least two vertical panels; at least two horizontal panels configured to form a lattice frame together with the at least two vertical panels, wherein each of the at least two horizontal panels is connected to each of the at least two vertical panels by an interior joint, respectively; and a feature configured to secure a mechanical component, an electrical component, or a plumbing component.

### CROSS-REFERENCE TO RELATED APPLICATION

This application claims priority under 35 U.S.C. § 119 to U.S. Provisional Application No. 63/346,010, filed on May 26, 2022, in the United States Patent and Trademark Office, the disclosure of which is incorporated by reference herein in its entirety.

### BACKGROUND

A residential home, such as a townhome, apartment, condominium, accessory dwelling unit, or single-family home (SFH), includes a complex network of building components. Examples of building components include a mechanical system, an electrical system, a plumbing system, and data infrastructure. Such systems provide basic conveniences found in a clean and comfortable home environment.

Mechanical, electrical, and plumbing (MEP) components and data infrastructure components are vital elements of many modern buildings. For instance, mechanical components may include heating and cooling (or temperature control) components, electrical components may include lights, outlets, appliances, etc., and plumbing components may include water lines, toilets, sinks, hook-ups, etc. In some cases, MEP components may be shipped and installed individually on-site during a construction process of a building. In some cases, MEP components may be installed in a building by different contractors at different times (e.g., with little to no communication on how the MEP components relate to one another).

In some cases, when one or more contractors build a home, the contractors might not consider tolerancing or constraints for secondary processes (e.g., as secondary processes may have been customized due to build tolerances). Timing and coordination of contractors to install the MEP components produce inherent inefficiencies. As a result, such builds may be ineffective, as there may be an excessive waste of material, contractor/build time, costs, etc. For example, extra furred-out walls may be required to address coordination deficiencies, resulting in reduced space and poor workflow. These costs are then passed to the homeowner or home buyer, contributing to already-excessive home affordability. There is therefore a need in the art for a modular wall component that increases the ease and efficiency of installing an MEP component in a building.

### SUMMARY

A modular wall component is described. One or more aspects of the modular wall component include at least two vertical panels; at least two horizontal panels configured to form a lattice frame together with the at least two vertical panels, wherein each of the at least two horizontal panels is connected to each of the at least two vertical panels by an interior joint, respectively; and a feature configured to secure a mechanical component, an electrical component, or a plumbing component.

A modular wall component is described. One or more aspects of the modular wall component include at least two vertical panels; at least two horizontal panels configured to form a lattice frame together with the at least two vertical panels, wherein each of the at least two horizontal panels forms an edge of a plurality of internal rectangular spaces defined by the at least two vertical panels; and a feature configured to secure a mechanical component, an electrical component, or a plumbing component.

A method for manufacturing a modular wall component is described. One or more aspects of the method, apparatus, non-transitory computer readable medium, and system include providing at least two vertical panels; providing at least two horizontal panels; cutting at least two interior joints in each of the at least two vertical panels and each of the at least two horizontal panels; cutting a feature from the at least two vertical panels or the at least two horizontal panels; and connecting the at least two vertical panels and the at least two horizontal panels using the at least two interior joints in each of the at least two vertical panels and the at least two horizontal panels to form the modular wall component.

### BRIEF DESCRIPTION OF THE DRAWINGS

FIG. 1 shows an example of a modular wall component according to aspects of the present disclosure.

FIG. 2 shows an example of a modular wall component including plumbing components according to aspects of the present disclosure.

FIG. 3 shows an example of a back view of the modular wall component of FIG. 2 according to aspects of the present disclosure.

FIG. 4 shows an example of a side view of the modular wall component of FIG. 2 according to aspects of the present disclosure.

FIG. 5 shows an example of a horizontal perspective cross-sectional view of the modular wall component of FIG. 2 according to aspects of the present disclosure.

FIG. 6 shows an example of a vertical cross-sectional view of the modular wall component of FIG. 2 according to aspects of the present disclosure.

FIG. 7 shows an example of a vertical perspective cross-sectional view of the modular wall component of FIG. 2 according to aspects of the present disclosure.

FIGS. 8 through 9 show examples of perspective detail views of the modular wall component of FIG. 2 according to aspects of the present disclosure.

FIG. 10 shows an example of a front detail view of the modular wall component of FIG. 2 according to aspects of the present disclosure.

FIG. 11 shows an example of a modular wall component installation according to aspects of the present disclosure.

FIGS. 12 through 15 show an example of installing a modular wall component according to aspects of the present disclosure.

FIG. 16 shows an example of a method for providing a modular wall component according to aspects of the present disclosure.

FIG. 17 shows an example of a method for providing a modular wall component including a second lattice frame according to aspects of the present disclosure.

### DETAILED DESCRIPTION

Mechanical, electrical, and plumbing (MEP) components and data infrastructure components are vital elements of many modern buildings. For instance, mechanical components may include heating and cooling (or temperature control) components, electrical components may include lights, outlets, appliances, etc., and plumbing components may include water lines, toilets, sinks, hook-ups, etc. In some cases, MEP components may be shipped and installed individually on-site during a construction process of a building. In some cases, MEP components may be installed in a building by different contractors at different times (e.g., with little to no communication on how the MEP components relate to one another).

In some cases, when one or more contractors build a home, the contractors might not consider tolerancing or constraints for secondary processes (e.g., as secondary processes may be customized due to build tolerances). As a result, such builds may be inefficient, as there may be an excessive waste of material, contractor/build time, costs, etc. For example, extra furred-out walls may be required to address coordination deficiencies, resulting in reduced space and poor workflow. As such, it may be desirable to design and manufacture an off-site-constructed modular unit that can be used in different-sized houses and in different locations.

According to some aspects, a modular wall component as described herein includes a lattice frame comprising interfacing panels to promote strength and ease of manufacturing. In some cases, the interfacing panels are cut from a wooden flat sheet stock, providing a three-dimensional structure in the general form of a lattice frame. In some cases, the modular wall component includes pre-located and pre-engineered mounting points for a mechanical component, an electrical component, a plumbing component, or a combination thereof. In some cases, the modular wall component includes the mechanical component, an electrical component, and/or a plumbing component. For example, a mechanical component may include a supply fan, an exhaust fan, an HVAC control system, condensation piping, ducting, a temperature sensor, a humidity sensor, a communication box, and the like. An electrical component may include an outlet, a light switch, solar conduit, a water heater whip, and the like. A plumbing component may include a toilet, hot/cold water supply, a valve, a shower temperature control, a drain line, a trap primer, an escutcheon, and the like.

In some cases, the modularity of the modular wall component provides for easy installation of the modular wall component and provides for other structures at the installation site to be considered and accounted for when designing and manufacturing the modular wall component. By contrast, during a traditional building process, a plumber, an electrician, an HVAC technician, a finisher, etc., may individually place each MEP component without a pre-existing structure to coordinate the installation of the MEP components, thereby risking damage and cross-contamination of componentry, etc. According to some aspects, the modular wall component provides a “plug-in” system that promotes an easy installation of one or more MEP components in the modular wall component according to predetermined locations.

In some cases, the modular wall component may provide an attachment system for an external component, such as a sink, a shower, a cabinet, etc. The modular wall component may be a plug-and-play system, such that on-site installation is minimally invasive and repeatable, and may include components such as plumbing components, electrical components, heating components, valves, switches, and the like. In some cases, the modular wall component can include a fire detection system, a fire protection system (e.g., such as sprinklers, water delivery systems, etc.), or a combination thereof.

In some cases, the modular wall component accounts for alignment on services like plumbing component slope indexing. In some cases, the modular wall component accounts for an affordance for a vertical movement of service sub-assemblies from an indexing point of a plumbing component to accommodate field connections to the plumbing component. In some cases, the modular wall component accounts for an affordance for an addition of structural reinforcement for load attachment points of the modular wall component. In some cases, the modular wall component provides for a field tolerance for connecting the modular wall component to a floor, a ceiling, a wall, or a combination thereof to be accommodated. In some cases, the modular wall component includes one or more features that provides for a “drop-in” of preconstructed MEP system sub-assemblies instead of a “fish-through” method of standard construction techniques.

### Modular Wall Component

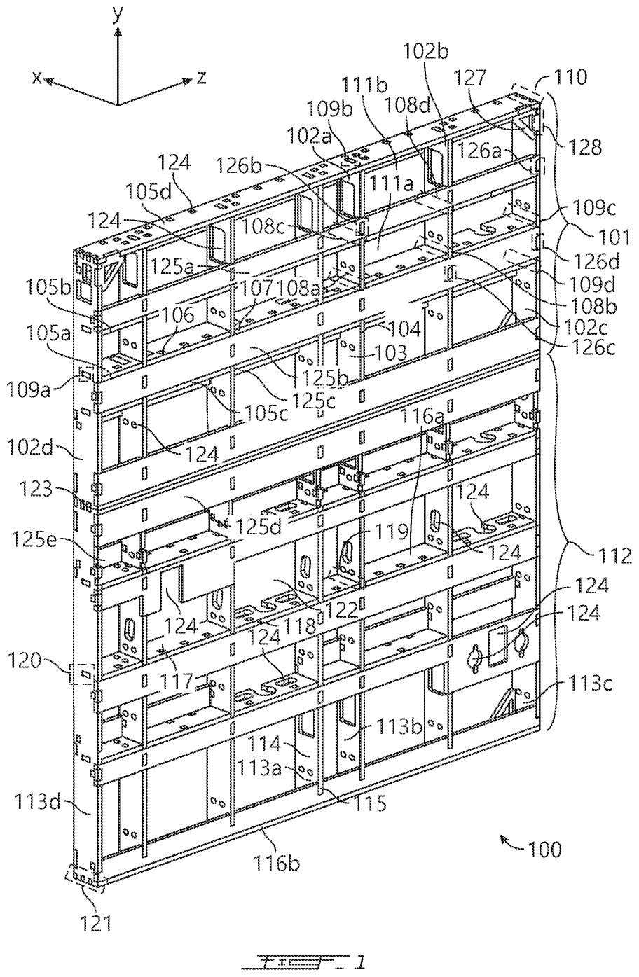

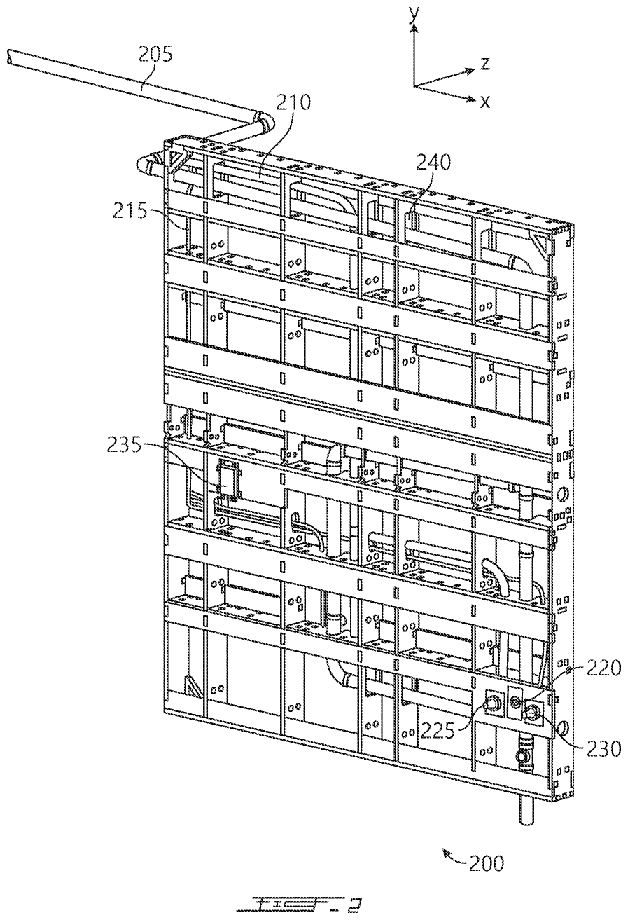

FIG. 1 shows an example of a modular wall component 100 according to aspects of the present disclosure. Modular wall component 100 is an example of, or includes aspects of, the corresponding element described with reference to FIGS. 2-15.

In one aspect, modular wall component 100 includes first lattice frame 101, second lattice frame 112, split 123, a plurality of features 124, first mounting panel 125a, second mounting panel 125b, third mounting panel 125c, fourth mounting panel 125d, fifth mounting panel 125e, first mounting panel joint 126a, second mounting panel joint 126b, third mounting panel joint 126c, fourth mounting panel joint 126d, first buttress 127, and buttress joint 128.

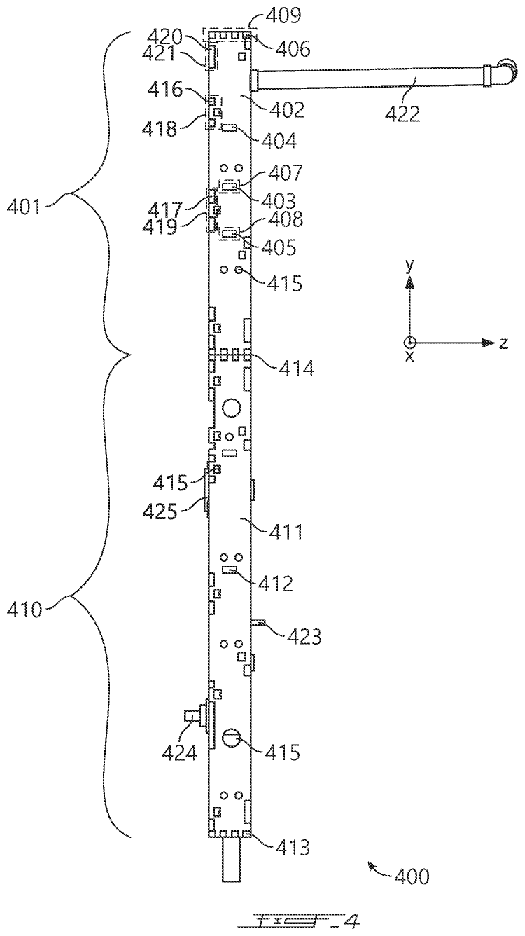

First lattice frame 101 is an example of, or includes aspects of, the corresponding element described with reference to FIG. 4. In one aspect, first lattice frame 101 includes first vertical panel 102a, second vertical panel 102b, third vertical panel 102c, fourth vertical panel 102d, first horizontal panel 105a, second horizontal panel 105b, third horizontal panel 105c, fourth horizontal panel 105d, first interior joint 108a, second interior joint 108b, third interior joint 108c, fourth interior joint 108d, fifth interior joint 109a, sixth interior joint 109b, seventh interior joint 109c, eighth interior joint 109d, ninth interior joint 110, first internal rectangular space 111a, and second internal rectangular space 111b.

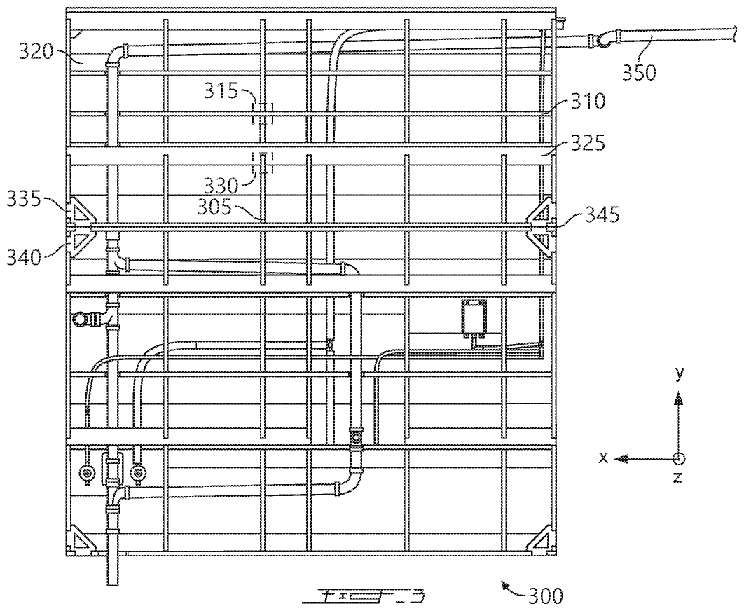

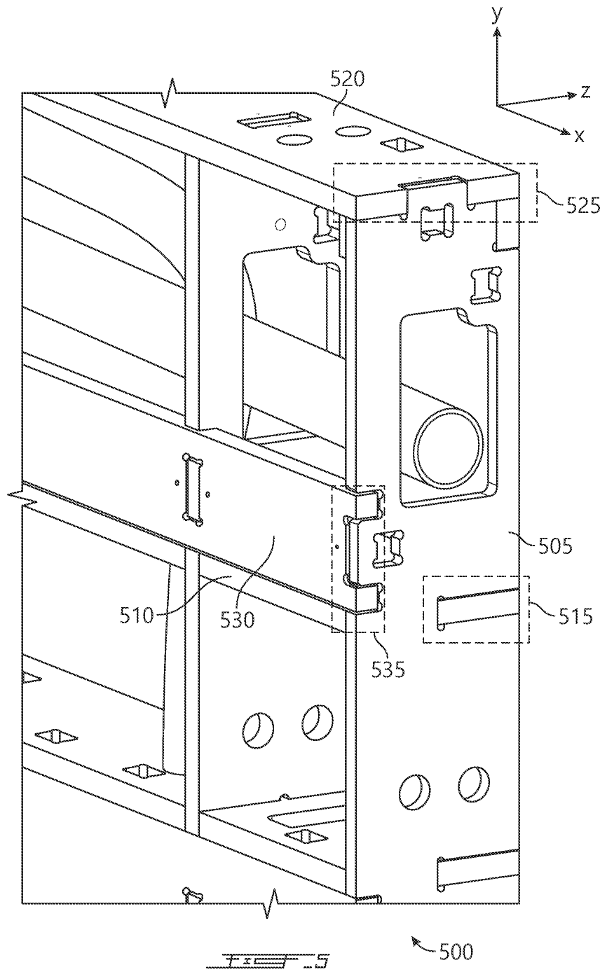

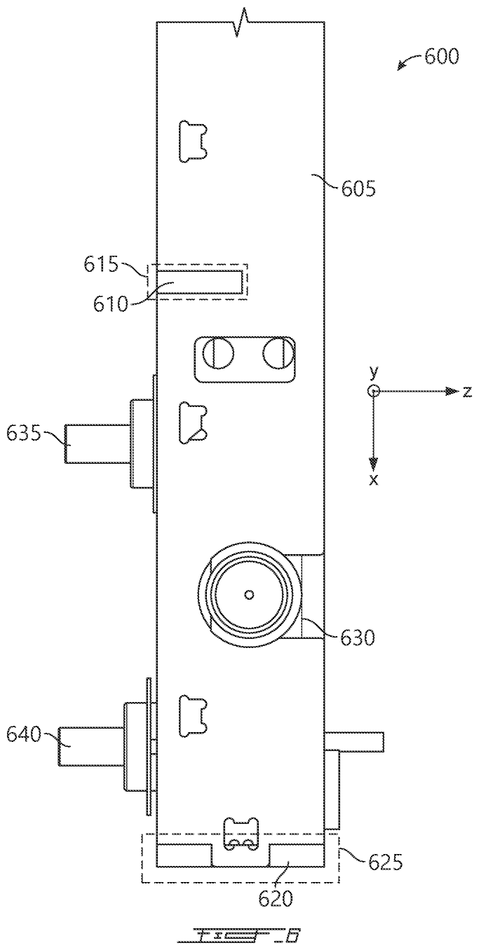

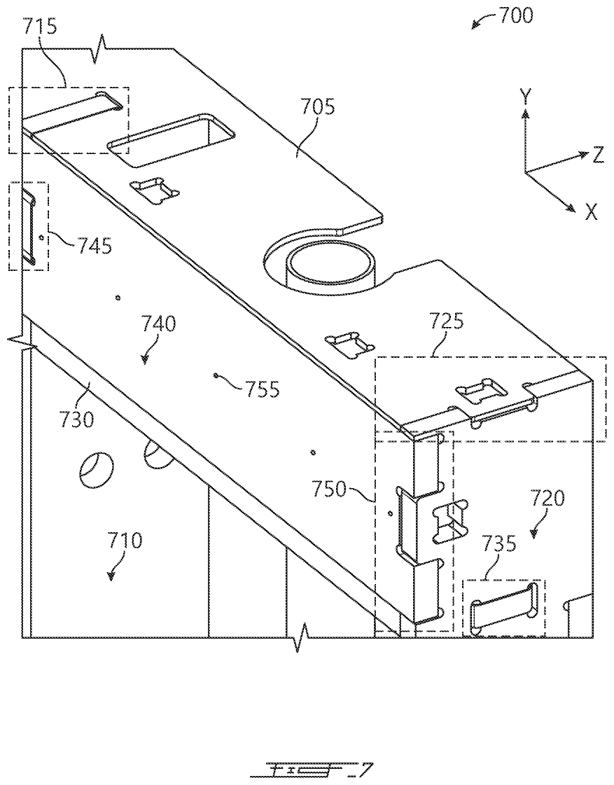

First vertical panel 102a is an example of, or includes aspects of, the corresponding element described with reference to FIGS. 3 and 5. Second vertical panel 102b is an example of, or includes aspects of, the corresponding element described with reference to FIGS. 6 and 7. Third vertical panel 102c is an example of, or includes aspects of, the corresponding element described with reference to FIGS. 4, 6, and 7. In one aspect, first vertical panel 102a includes vertical interior face 103 and vertical exterior face 104.

As shown in FIG. 1, each of first vertical panel 102a, second vertical panel 102b, third vertical panel 102c, and fourth vertical panel 102d is an example of a vertical panel included in a set of vertical panels included in first lattice frame 101. As shown in FIG. 1, first lattice frame 101 includes seven vertical panels. However, embodiments of the present disclosure are not limited thereto, and a lattice frame according to the present disclosure may include one or more vertical panels.

According to some aspects, a vertical panel extends in a first direction (e.g., a y-direction). In some cases, a vertical interior face (such as vertical interior face 103) of a vertical panel faces a second direction crossing the first direction (e.g., an x-direction). In some cases, the second direction is orthogonal to the first direction. In some cases, a vertical exterior face (such as vertical exterior face 104) of a vertical panel faces a third direction crossing the first direction and the second direction (e.g., a z-direction). In some cases, the third direction is orthogonal to the first direction and to the second direction.

In some cases, a vertical interior face of a vertical panel (e.g., first vertical panel 102a) faces another vertical interior face of another vertical panel (e.g., second vertical panel 102b). In some cases, a vertical exterior face (e.g., vertical exterior face 104) of a vertical panel faces outwardly from modular wall component 100 in the third direction.

According to some aspects, a vertical panel comprises a wood. According to some aspects, a vertical panel comprises at least one of a wood, a wood composite, a plastic, a metal, or a masonry material (including, e.g., concrete or brick).

First horizontal panel 105a is an example of, or includes aspects of, the corresponding element described with reference to FIGS. 3-4 and 6-7. Second horizontal panel 105b is an example of, or includes aspects of, the corresponding element described with reference to FIGS. 4 and 5. Third horizontal panel 105c is an example of, or includes aspects of, the corresponding element described with reference to FIGS. 4 and 7. Fourth horizontal panel 105d is an example of, or includes aspects of, the corresponding element described with reference to FIGS. 4 and 5. In one aspect, first horizontal panel 105a includes horizontal interior face 106 and horizontal exterior face 107.

As shown in FIG. 1, each of first horizontal panel 105a, second horizontal panel 105b, third horizontal panel 105c, and fourth horizontal panel 105d is an example of a horizontal panel of a set of horizontal panels included in first lattice frame 101. As shown in FIG. 1, first lattice frame 101 includes five horizontal panels. However, embodiments of the present disclosure are not limited thereto, and a lattice frame according to the present disclosure may include one or more horizontal panels.

According to some aspects, a horizontal panel extends in the second direction. In some cases, a horizontal interior face (such as horizontal interior face 106) of a horizontal panel faces the first direction. In some cases, a horizontal exterior face (such as horizontal exterior face 107) of a horizontal panel faces the third direction.

In some cases, a horizontal interior face of a horizontal panel (e.g., first horizontal panel 105a) faces another horizontal interior face of another horizontal panel (e.g., second horizontal panel 105b). In some cases, a horizontal exterior face (e.g., horizontal exterior face 107) of a horizontal panel faces outwardly from modular wall component 100 in the third direction.

According to some aspects, a horizontal panel comprises a wood. According to some aspects, a horizontal panel comprises at least one of a wood, a wood composite, a plastic, a metal, or a masonry material (including, e.g., concrete or brick).

First interior joint 108a is an example of, or includes aspects of, the corresponding element described with reference to FIG. 3. Second interior joint 108b is an example of, or includes aspects of, the corresponding element described with reference to FIGS. 6 and 7. Third interior joint 108c and sixth interior joint 109b are examples of, or include aspects of, the corresponding elements described with reference to FIG. 5. Seventh interior joint 109c is an example of, or includes aspects of, the corresponding element described with reference to FIGS. 4 and 6-7. Eighth interior joint 109d is an example of, or includes aspects of, the corresponding element described with reference to FIGS. 4 and 7. Ninth interior joint 110 is an example of, or includes aspects of, the corresponding element described with reference to FIG. 4.

In some aspects, at least two horizontal panels (e.g., first horizontal panel 105a and second horizontal panel 105b) are configured to form the first lattice frame 101 together with at least two vertical panels (e.g., first vertical panel 102a and second vertical panel 102b), where each of the at least two horizontal panels is connected to each of the at least two vertical panels by an interior joint (e.g., first interior joint 108a, second interior joint 108b, third interior joint 108c, and fourth interior joint 108d), respectively. In some aspects, an interior joint includes a slot joint.

For example, in some cases, a vertical panel (such as first vertical panel 102a) extends such that the vertical panel contacts at least two horizontal panels (e.g., first horizontal panel 105a and second horizontal panel 105b). Likewise, in some cases, a horizontal panel (such as first horizontal panel 105a) extends such that the horizontal panel contacts at least two vertical panels (e.g., first vertical panel 102a and second vertical panel 102b). In some cases, at each point of contact between a vertical panel and a horizontal panel, the vertical panel and the horizontal panel form an interior joint.

In some cases, an “interior joint” (such as first interior joint 108a, second interior joint 108b, third interior joint 108c, fourth interior joint 108d, fifth interior joint 109a, sixth interior joint 109b, seventh interior joint 109c, eighth interior joint 109d, or ninth interior joint 110) refers to a joint that may be formed by two or more panels such that an interior face of a first panel is adjacent to and forms an angle (such as a right angle) with an interior face of a second panel. In an example, as shown in FIG. 1, first interior joint 108a is formed by first vertical panel 102a and first horizontal panel 105a such that vertical interior face 103 is disposed adjacent to and forms an angle with a horizontal interior face of first horizontal panel 105a.

In some cases, each of first interior joint 108a, second interior joint 108b, third interior joint 108c, fourth interior joint 108d, fifth interior joint 109a, sixth interior joint 109b, seventh interior joint 109c, eighth interior joint 109d, and ninth interior joint 110 is a slot joint. In some cases, a “slot joint” refers to a joint that may be formed by inserting a slotted portion of a first panel into a slotted portion of at least a second panel. For example, as shown in FIG. 1, first interior joint 108a is formed as a slot joint by inserting a slot disposed in first vertical panel 102a into a slot disposed in first horizontal panel 105a, such that at least a portion of first vertical panel 102a at least partially surrounds at least a portion of first horizontal panel 105a, and at least a portion of first horizontal panel 105a at least partially surrounds at least a portion of first vertical panel 102a.

In some cases, a slot joint comprises a middle joint (such as fifth interior joint 109a). In some cases, a “middle joint” refers to a joint that may be formed by at least two panels, where at least one tab of the first panel (e.g., a portion of the first panel that extends from an edge of the first panel) is respectively inserted into at least one slot of at least the second panel, such that the at least one tab is at least partially enclosed (e.g., on four sides) by the second panel. For example, as shown in FIG. 1, fifth interior joint 109a (indicated by dashed lines) is formed as a middle joint by inserting a tab of first horizontal panel 105a that extends in the second direction from an edge of first horizontal panel 105a into a slot disposed in fourth vertical panel 102d, and sixth interior joint 109b (indicated by dashed lines) is formed by inserting a tab of first vertical panel 102a that extends in the first direction from an edge of first vertical panel 102a into a slot disposed in fourth horizontal panel 105d.

In some cases, a slot joint comprises an end joint (such as ninth interior joint 110). In some cases, an “end joint” refers to a joint that may be formed by at least two panels, where at least one tab of the first panel (e.g., a portion of the first panel that extends from an edge of the first panel) is respectively inserted into at least one slot of at least the second panel, where the slot is disposed at an edge of the second panel, such that the at least one tab is at least partially enclosed (e.g., on at least three sides) by the second panel. From another structurally equivalent perspective, in some cases, an end joint may be formed by inserting a tab extending from the first panel between a pair of tabs extending from the second panel. For example, as shown in FIG. 1, ninth interior joint 110 (indicated by dashed lines) is formed by inserting a tab of third vertical panel 102c that extends in the first direction from an edge of third vertical panel 102c into a slot disposed at an edge of fourth horizontal panel 105d, or by inserting a tab of fourth horizontal panel 105d that extends in the second direction from an edge of fourth horizontal panel 105d into a slot disposed at an edge of third vertical panel 102c.

In some aspects, at least two horizontal panels are configured to form a lattice frame (e.g., first lattice frame 101) together with at least two vertical panels, where each of the at least two horizontal panels forms an edge of a set of internal rectangular spaces (including, for example, first internal rectangular space 111a and second internal rectangular space 111b) defined by the at least two vertical panels. For example, as shown in FIG. 1, first internal rectangular space 111a is defined (e.g., at least partially surrounded) by a pair of opposing vertical interior faces of first vertical panel 102a and second vertical panel 102b, respectively, and a pair of opposing horizontal interior faces of first horizontal panel 105a and second horizontal panel 105b, respectively. Likewise, as shown in FIG. 1, second internal rectangular space 111b is defined (e.g., at least partially surrounded) by a pair of opposing vertical interior faces of first vertical panel 102a and second vertical panel 102b, respectively, and a pair of opposing horizontal interior faces of second horizontal panel 105b and fourth horizontal panel 105d, respectively.

According to some aspects, a vertical panel and a horizontal panel may be retained, adhered, joined, and/or connected to each other by one or more of a joint, a screw, a bolt, an adhesive, a magnet, a friction fit system, a bracket, etc. A friction fit system, or interference fit, is a form of fastening using at least two tightly mating parts or features to adhere the features by use of friction. In some cases, a friction fit system may include a crush system including a crusher feature or crushing feature. The crusher feature may include a dimension that is larger than a crushing feature and may deform the crusher or crushing feature, resulting in a lasting friction fit system.

In some cases, a lattice frame (e.g., first lattice frame 101) includes an outermost vertical panel (e.g., third vertical panel 102c or fourth vertical panel 102d) that might not be disposed between a pair of vertical panels in the second direction. In some cases, an outermost vertical panel includes an outermost vertical face that faces away from modular wall component 100 in the second direction. In some cases, an outermost vertical panel forms an interior joint (such as a middle joint, e.g., fifth interior joint 109a) with a horizontal panel.

In some cases, a lattice frame (e.g., first lattice frame 101) includes an outermost horizontal panel (e.g., fourth horizontal panel 105d) that might not be disposed between a pair of horizontal panels in the first direction. In some cases, the outermost horizontal panel includes an outermost horizontal face that faces away from modular wall component 100 in the first direction. In some cases, the outermost horizontal panel forms an interior joint (such as a middle joint, e.g., sixth interior joint 109b) with a vertical panel.

In some cases, an outermost vertical panel (e.g., third vertical panel 102c) forms an interior joint (such as an end joint, e.g., ninth interior joint 110) with an outermost horizontal panel (e.g., fourth horizontal panel 105d). In some cases, a pair of outermost vertical panels and a pair of outermost horizontal panels partially surrounds a set of vertical panels and a set of horizontal panels to form a lattice frame (e.g., first lattice frame 101).

As shown in FIG. 1, modular wall component 100 includes two lattice frames (e.g., first lattice frame 101 and second lattice frame 112). However, embodiments of the present disclosure are not limited thereto, and modular wall component 100 can include various numbers of lattice frames (such as one or more than two) oriented and/or split according to various installation specifications.

Second lattice frame 112 is an example of, or includes aspects of, the corresponding element described with reference to FIG. 4. A repeated description of one or more similar elements is omitted for the sake of brevity.

According to some aspects, a lattice frame (e.g., first lattice frame 101 or second lattice frame 112) is arranged as a torsion box frame. In some cases, the lattice frame provides strength and stiffness in the first (e.g., a vertical) direction, the second (e.g., a horizontal) direction, and the third (e.g., a shear) direction of modular wall component 100. According to some aspects, a width in the third direction of a vertical panel is equal to a width in the third direction of a horizontal panel and a depth of the lattice frame. According to some aspects, the depth of the lattice frame in the third direction is in the range of about three inches (e.g., three inches) to about five feet (e.g., five feet). According to some aspects, the depth of the lattice frame in the third direction is determined according to a mechanical component, an electrical component, or a plumbing component to be disposed in modular wall component 100. According to some aspects, the depth of the lattice frame in the third direction is determined according to an external structure. According to some aspects, the depth of the lattice frame in the third direction is determined according to a standard lumber dimension.

In one aspect, second lattice frame 112 includes first additional vertical panel 113a, second additional vertical panel 113b, third additional vertical panel 113c, fourth additional vertical panel 113d, first additional horizontal panel 116a, second additional horizontal panel 116b, first additional interior joint 119, second additional interior joint 120, third additional interior joint 121, and additional internal rectangular space 122.

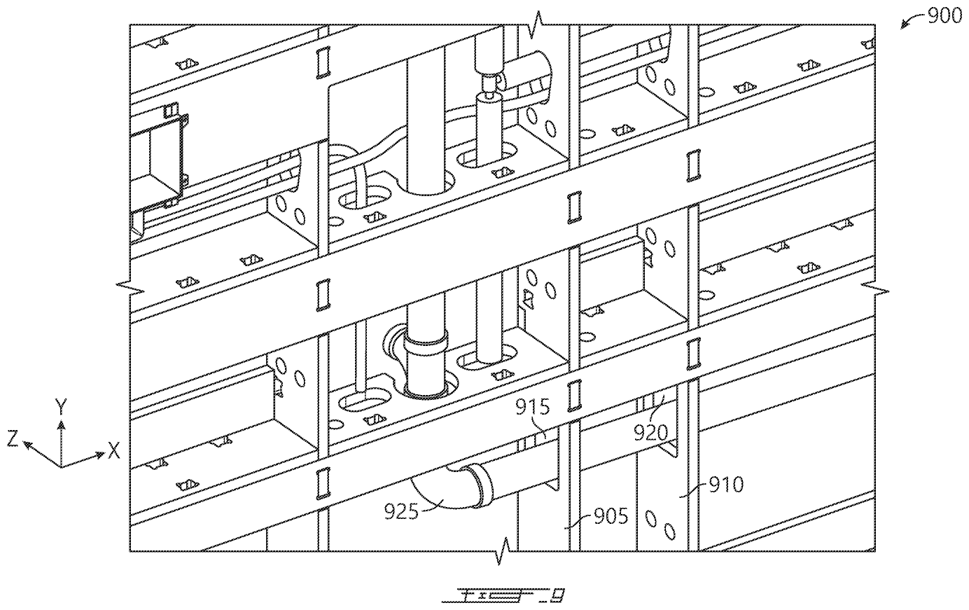

First additional vertical panel 113a and second additional vertical panel 113b are examples of, or include aspects of, the corresponding elements described with reference to FIG. 9. Third additional vertical panel 113c is an example of, or includes aspects of, the corresponding element described with reference to FIG. 4. In one aspect, first additional vertical panel 113a includes additional vertical interior face 114 and additional vertical exterior face 115. First additional horizontal panel 116a and second additional horizontal panel 116b is an example of, or includes aspects of, the corresponding elements described with reference to FIG. 4. In one aspect, first additional horizontal panel 116a includes additional horizontal interior face 117 and additional horizontal exterior face 118.

According to some aspects, second lattice frame 112 comprises at least two additional vertical panels (e.g., first additional vertical panel 113a and second additional vertical panel 113b) and at least two additional horizontal panels (e.g., first additional horizontal panel 116a and second additional horizontal panel 116b), where each of the at least two additional horizontal panels is connected to each of the at least two additional vertical panels by an interior joint (e.g., first additional interior joint 119, second additional interior joint 120, or third additional interior joint 121), respectively.

As shown in FIG. 1, first additional interior joint 119 (indicated by dashed lines) is a slot joint formed by first additional vertical panel 113a and first additional horizontal panel 116a, second additional interior joint 120 (indicated by dashed lines) is a middle joint formed by first additional horizontal panel 116a and fourth additional vertical panel 113d, and third additional interior joint 121 (indicated by dashed lines) is an end joint formed by second additional horizontal panel 116b and fourth additional vertical panel 113d.

According to some aspects, an additional vertical panel and an additional horizontal panel may be retained, adhered, joined, and/or connected to each other by one or more of a joint, a screw, a bolt, an adhesive, a magnet, a friction fit system, a bracket, etc.

As shown in FIG. 1, second lattice frame 112 is joined to first lattice frame 101 to form modular wall component 100. In some cases, second lattice frame 112 is joined to first lattice frame 101 via a joint (such as a slot joint, a lap joint, a cross-lap joint, a 3D finger tenon joint, etc.). In some cases, second lattice frame 112 is jointed to first lattice frame 101 via a screw, a bolt, an adhesives, a magnet, friction, or the like. In some cases, first lattice frame 101 and second lattice frame 112 are joined via a friction-fit system, and may be interlocked into one another via a joint system. According to some aspects, a depth of second lattice frame 112 in the third direction is equal to or different than the depth of first lattice frame 101 in the third direction.

Split 123 is an example of, or includes aspects of, the corresponding element described with reference to FIGS. 3-4. In some cases, a split between two or more lattice frames (such as split 123) may be a flat-planed interface (e.g., one or more points of contact) between the two or more lattice frames (e.g., first lattice frame 101 and second lattice frame 112). In some examples, a split (e.g., an interface) is a vertical split in which a vertical panel of a first lattice frame is joined to an additional vertical panel of a second lattice frame. In some examples, a split is a horizontal split in which a horizontal panel of a first lattice frame is joined to an additional horizontal panel of a second lattice frame. In some cases, a split (e.g., an interface) may comprise a curved or a three-dimensional interface. In some examples, a split is a split in the depth direction in which one or more of a vertical exterior face and a horizontal exterior face of a first lattice frame is joined to one or more of an additional vertical exterior face and an additional horizontal exterior face of a second lattice frame. As shown in FIG. 1, split 123 is a horizontal split in which a horizontal panel of first lattice frame 101 is joined to an additional horizontal panel of second lattice frame 112.

Accordingly, in some aspects, a modular wall component such as modular wall component 100 may be provided as individual boxes/pieces/frames (e.g., a combination of components including one or more lattice frames such as first lattice frame 101 and second lattice frame 112) that can be separated from one another and joined to one another at one or more splits (such as split 123). The modularity of the modular wall component provides the ability for one or more pieces of the modular wall component to be carried or transferred through door openings, window openings, stairs, and the like. Therefore, the modular wall component can be designed and manufactured in ever-smaller modular components that can account for installation constraints and/or external factors specific to an installation setting.

In some cases, a split is oriented based on a geometry of an external structure, on a weight, size, and/or shape of one or more elements of the modular wall component, a geometry of an installation site of the modular wall component, or a combination thereof. For example, the modular wall component may be split based on installation factors such as doorways used to access an installation site of the modular wall component, or on external factors specific to the installation site such as shapes, sizes, and/or locations of external components (e.g., sinks, toilets, washers, dryers, refrigerators, external water lines, external electrical lines, etc.), aspects of adjoining/adjacent walls installed or to be installed, aspects of adjoining/adjacent ceiling installed or to be installed, aspects of adjoining/adjacent floors installed or to be installed, additional componentry and devices installed or to be installed, finish componentry installed or to be installed (including sinks, shower, cabinets), etc.

In an example, a split is oriented based on a horizontal vent line in which a washer and a dryer tie into a main stack (which may be, for example, about eight inches above the counter height). As a result, in some cases, an electrical panel may be eight inches above the counter height. In some cases, an electrical panel may be lower than a standard height due to electronic componentry in the lower section.

According to some aspects, modular wall component 100 includes a plurality of features 124. However, embodiments of the present disclosure are not limited thereto, and a modular wall component may include one feature. In some cases, a feature of the plurality of features 124 is a slot, a hole, a void, etc. of variable shape, size, orientation, and/or location that penetrates at least partially through a vertical panel, a horizontal panel, an additional vertical panel, or an additional horizontal panel. In some cases, a feature of the plurality of features 124 is shaped, sized, oriented, and/or located to accommodate at least a portion of a mechanical component, an electrical component, a plumbing component, or a combination thereof in the feature. In some cases, the plurality of features 124 is configured to accommodate an alignment of a mechanical component, an electrical component, a plumbing component, an external component, or a combination thereof. The plurality of features 124 is an example of, or includes aspects of, the corresponding element described with reference to FIGS. 2, 4, and 7-9.

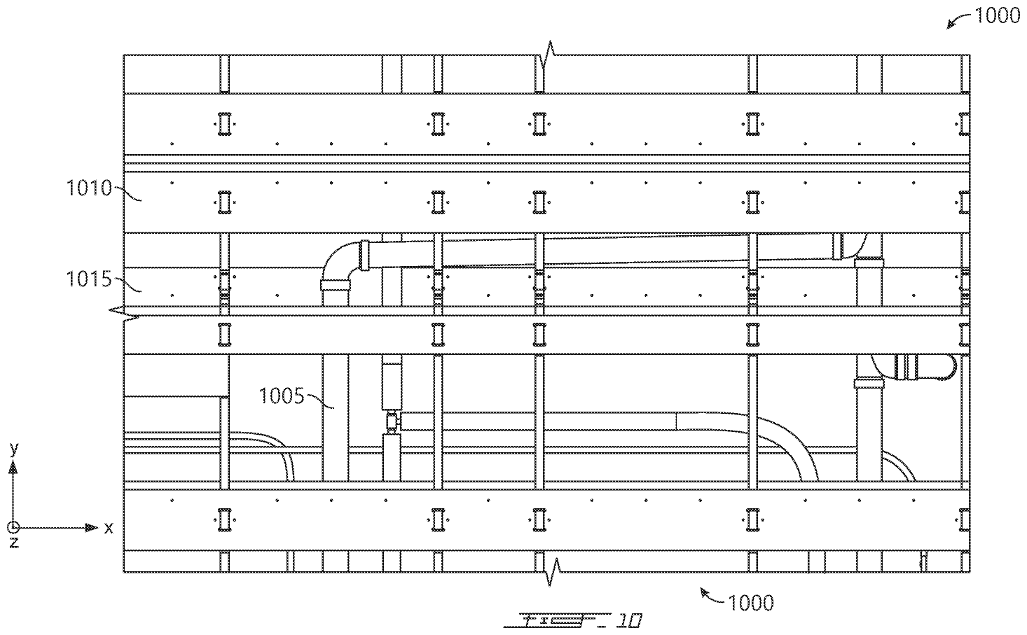

First mounting panel 125a is an example of, or includes aspects of, the corresponding element described with reference to FIGS. 3-5. Second mounting panel 125b is an example of, or includes aspects of, the corresponding element described with reference to FIGS. 4 and 7. Third mounting panel 125c is an example of, or includes aspects of, the corresponding element described with reference to FIG. 3. Fourth mounting panel 125d and fifth mounting panel 125e are examples of, or include aspects of, the corresponding elements described with reference to FIG. 10.

According to some aspects, each of first mounting panel 125a, second mounting panel 125b, third mounting panel 125c, fourth mounting panel 125d, and fifth mounting panel 125e is an example of a mounting panel of a set of mounting panels included in modular wall component 100. According to some aspects, a modular wall component may comprise one or more mounting panels. According to some aspects, a mounting panel is omitted from a modular wall component. In some cases, a mounting panel extends in the second direction. In some cases, a face of the mounting panel faces outwardly from modular wall component 100 in the third direction. According to some aspects, a mounting panel comprises a wood. According to some aspects, a mounting panel comprises at least one of a wood, a wood composite, a plastic, a metal, or a masonry material (including, e.g., concrete or brick).

First mounting panel joint 126a is an example of, or includes aspects of, the corresponding element described with reference to FIG. 4. Second mounting panel joint 126b is an example of, or includes aspects of, the corresponding element described with reference to FIG. 5. Third mounting panel joint 126c and fourth mounting panel joint 126d are examples of, or include aspects of, the corresponding elements described with reference to FIG. 7. Fourth mounting panel joint 126d is an example of, or includes aspects of, the corresponding element described with reference to FIGS. 4 and 7.

According to some aspects, a mounting panel is joined to an external face of a vertical panel or an additional vertical panel by a mounting panel joint (such as a slot joint, including a middle joint or an end joint). For example, as shown in FIG. 1, first mounting panel 125a is joined to first vertical panel 102a by second mounting panel joint 126b, and first mounting panel 125a is joined to third vertical panel 102c by first mounting panel joint 126a.

According to some aspects, a mounting panel joint may be formed by inserting a tab extending from a vertical panel or an additional vertical panel into a slot of a mounting panel. For example, as shown in FIG. 1, first mounting panel joint 126a is formed by inserting a tab extending from third vertical panel 102c into a slot of first mounting panel 125a, second mounting panel joint 126b is formed by inserting a tab extending from first vertical panel 102a into a slot of first mounting panel 125a, third mounting panel joint 126c is formed by inserting a tab extending from second vertical panel 102b into a slot of second mounting panel 125b, and fourth mounting panel joint 126d is formed by inserting a tab extending from third vertical panel 102c into a slot of second mounting panel 125b. According to some aspects, a mounting panel can be similarly joined to a horizontal panel or an additional horizontal panel.

According to some aspects, a mounting panel may be retained, adhered, joined, and/or connected to one or more of a vertical panel, a horizontal panel, an additional vertical panel, or an additional horizontal panel by one or more of a joint, a screw, a bolt, an adhesive, a magnet, a friction fit system, a bracket, or the like. For example, in some cases, a mounting panel and the one or more of a vertical panel, a horizontal panel, an additional vertical panel, or an additional horizontal panel are joined via a friction-fit system, and may be interlocked into one another via a joint system.

According to some aspects, at least a portion of a vertical panel or an additional vertical panel overlaps at least a portion of a mounting panel in the third direction. For example, as shown in FIG. 1, a portion of first vertical panel 102a overlaps first mounting panel 125a in the third direction. According to some aspects, at least a portion of a horizontal panel or an additional horizontal panel overlaps at least a portion of a mounting panel in the third direction. For example, as shown in FIG. 1, a portion of first horizontal panel 105a overlaps second mounting panel 125b in the third direction. For example, according to some aspects, an outwardly facing surface of a mounting panel in the third direction is flush in the first direction with a vertical panel and/or a horizontal panel.

In some cases, one or more features are disposed in a mounting panel and penetrate at least partially through the mounting panel. In some cases, a mounting panel comprises a load attachment point. For example, in some cases, a mechanical component, an electrical component, a plumbing component, or an external structure (such as a cabinet, an appliance, etc.) is attached to the mounting panel. According to some aspects, a mounting panel is configured to be joined to a finish siding (such as plywood, drywall, etc.) to provide a finishing side for modular wall component 100.

In some cases, a feature of the plurality of features 124 includes a shelf alignment hole used to locate a shelf without measuring. In an example, a pair of features of the plurality of features 124 are implemented as pilot holes that may be predrilled into a mounting panel to locate a shelf to be installed onto modular wall component 100. In the example, after one screw is installed into one of the features, the other feature is aligned, and the second screw is installed into the other feature, thereby avoiding use of a tape measure and a level by the installer of the shelf.

According to some aspects, a feature of the plurality of features 124 is embedded in one or more of a vertical panel, a horizontal panel, an additional vertical panel, an additional horizontal panel, or a mounting panel. For example, in some cases, a numerical control (NC) program is used to calculate and determine locations of one or more features of the plurality of features 124. In some cases, a computer numerical control (CNC) machine uses the NC program to locate and subtract the one or more features from one or more of a vertical panel, a horizontal panel, an additional vertical panel, an additional horizontal panel, or a mounting panel. In some cases, a feature of the plurality of features 124 is formed using an additive manufacturing process, a subtractive manufacturing process, or a combination thereof.

According to some aspects, one or more of a vertical panel, a horizontal panel, an additional vertical panel, an additional horizontal panel, or a mounting panel are formed via a subtractive process, injection molding, 3D printing, or any other suitable manufacturing method.

According to some aspects, a vertical panel can be interconnected with a horizontal panel via a screw, a bolt, adhesive, a magnet, friction, or the like. According to some aspects, an additional vertical panel can be interconnected with an additional horizontal panel via a screw, a bolt, adhesive, a magnet, friction, or the like. According to some aspects, a mounting panel can be interconnected with a vertical panel, a horizontal panel, an additional vertical panel, an additional horizontal panel, or a combination thereof via a screw, a bolt, adhesive, a magnet, friction, or the like.

According to some aspects, one or more of an interior joint, an additional interior joint, or a mounting panel joint is implemented as a slot joint, a lap joint, a cross-lap joint, a 3D finger tenon joint, or any other suitable joint.

First buttress 127 and buttress joint 128 are examples of, or include aspects of, the corresponding elements described with reference to FIG. 4. According to some aspects, a buttress (such as first buttress 127) contacts at least two of a group including a vertical panel, a horizontal panel, an additional vertical panel, and an additional horizontal panel. According to some aspects, a buttress is joined to one or more of a vertical panel, a horizontal panel, an additional vertical panel, and an additional horizontal panel 116 via a buttress joint (such as buttress joint 128). In some cases, a buttress joint is a slot joint (for example, a slot joint, a middle joint, or an end joint). In some cases, a buttress (e.g., first buttress 127) provides additional structural rigidity for modular wall component 100. In some cases, a buttress comprises a wood, a wood composite, a plastic, a metal, a masonry material (including, e.g., concrete or brick), or a combination thereof. In some cases, a buttress (e.g., first buttress 127) and/or a buttress joint (e.g., buttress joint 128) are included in one or more lattice frames (e.g., first lattice frame 101 and/or second lattice frame 112). First buttress 127 and buttress joint 128 are examples of, or include aspects of, the corresponding elements described with reference to FIG. 4.

According to some aspects, modular wall component 100 can be at least partially enclosed on one or more exterior sides that face away from modular wall component 100 in the third direction (for example, by attaching a facade panel, such as plywood, to a vertical exterior face 104, a horizontal exterior face 107, an additional vertical exterior face 115, an additional horizontal exterior face 118, a mounting panel 125, or a combination thereof).

According to some aspects, modular wall component 100 includes an integrated pickup for a crane attachment. As a result, a crane or secondary equipment can pick up and place modular wall component 100 (e.g., for installation). According to some aspects, modular wall component 100 includes backing for American Disabilities Act (ADA) components and bracketry. According to some aspects, modular wall component 100 includes integrated attachment points for attaching modular wall component 100 to a ceiling, a wall, and/or a floor.

FIG. 2 shows an example of a modular wall component 200 including plumbing components according to aspects of the present disclosure. Modular wall component 200 is an example of, or includes aspects of, the corresponding element described with reference to FIGS. 1, and 3-15. A repeated description of one or more similar elements is omitted for the sake of brevity.

According to some aspects, modular wall component 200 is an integrated assembly that can function as both a wall and as an MEP containment structure. In some cases, items such as water lines, outlets, a water heater, and the like can be preinstalled in modular wall component 200, and modular wall component 200 can be shipped and installed as a single unit. In some examples, modular wall component 200 can be installed or located behind a kitchen, a bathroom, a laundry room, or the like. For example, in some cases, modular wall component 200 can function as a single wall structure disposed between a bathroom and a kitchen, and modular wall component 200 can include MEP componentry to support the bathroom and the kitchen (e.g., to support external bathroom and kitchen components such as sinks, toilets, stove tops, refrigerators, etc.).

According to some aspects, one or more of a mechanical component, an electrical component, or a plumbing component is attached to an element of modular wall component 200 via an attachment mechanism such as a bracket, a bolt, a screw, an adhesive, a magnet, or any other suitable attachment mechanism. According to some aspects, the attachment mechanism is placed in or on modular wall component 200 at a predetermined location.

In one aspect, modular wall component 200 includes first plumbing component 205, second plumbing component 210, third plumbing component 215, fourth plumbing component 220, fifth plumbing component 225, sixth plumbing component 230, seventh plumbing component 235, and a plurality of features 240.

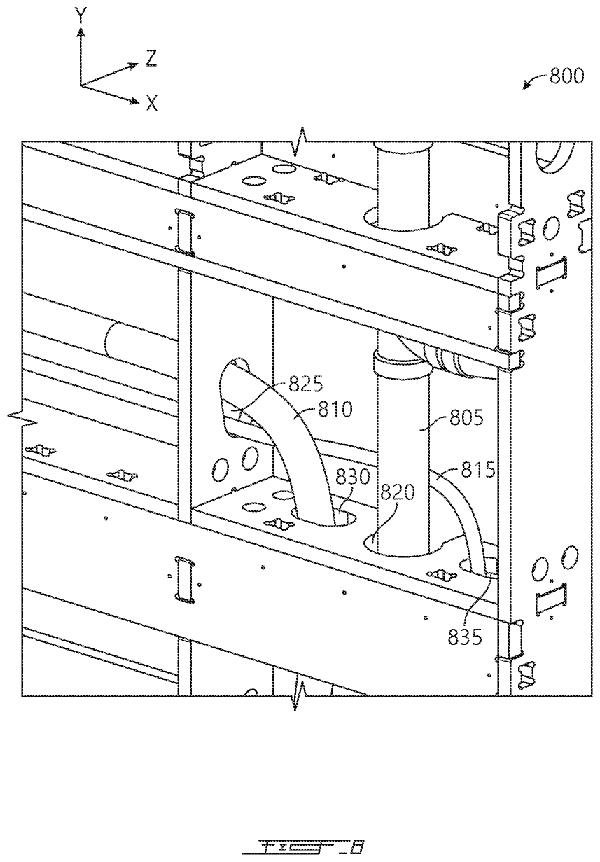

First plumbing component 205 is an example of, or includes aspects of, the corresponding element described with reference to FIGS. 3, 4, 6, and 8-10. Second plumbing component 210 is an example of, or includes aspects of, the corresponding element described with reference to FIGS. 4 and 8. Third plumbing component 215 is an example of, or includes aspects of, the corresponding element described with reference to FIG. 8. Fifth plumbing component 225 is an example of, or includes aspects of, the corresponding element described with reference to FIGS. 4 and 6. Sixth plumbing component 230 is an example of, or includes aspects of, the corresponding element described with reference to FIG. 6. Seventh plumbing component 235 is an example of, or includes aspects of, the corresponding element described with reference to FIG. 7. The plurality of features 240 is an example of, or includes aspects of, the corresponding element described with reference to FIGS. 1, 4, 7, and 8.

Referring to FIG. 2, first plumbing component 205, second plumbing component 210, third plumbing component 215, fourth plumbing component 220, fifth plumbing component 225, sixth plumbing component 230, and seventh plumbing component 235 are respectively partially disposed in the plurality of features 240. However, embodiments of the present disclosure are not limited thereto, and according to some aspects, one or more of a mechanical component, an electrical component, a plumbing component, or a combination thereof are disposed in modular wall component 200 and/or the plurality of features 240.

As shown in FIG. 2, each of first plumbing component 205, second plumbing component 210, and third plumbing component 215 are a water line or a pipe, each of fourth plumbing component 220, fifth plumbing component 225, and sixth plumbing component 230 is a connecting component for respectively connecting an external component to first plumbing component 205, second plumbing component 210, and third plumbing component 215, and seventh plumbing component 235 is a water box for a refrigerator.

According to some aspects, the plurality of features 240 are located in modular wall component 200 to provide readily identifiable for positions of a mechanical component, an electrical component, a plumbing component, or a combination thereof in modular wall component 200. In some cases, a layout of the plurality of features 240 is determined according to a planned layout for the mechanical component, the electrical component, the plumbing component, or the combination thereof withing modular wall component 200. In some cases, one or more features of the plurality of features 240 provides an ability for modular wall component 200 to restrain movement of the mechanical component, the electrical component, the plumbing component to a predetermined range and location.

According to some aspects, modular wall component 200 provides for a plumbing component (such as a water line) to be set back from an exterior wall of a building to avoid possible damage that may be caused by the use of long screws. In some cases, modular wall component 200 includes an embedded or integrated water shut-off valve with access through an access hole/panel. In some cases, the access may be through a kitchen cabinet sink. In some cases, modular wall component 200 includes washer and dryer connections.

According to some aspects, a plumbing component including cross-linked polyethylene (PEX) is included in modular wall component 200, but the present disclosure is not limited thereto. For example, copper or any other suitable material for a plumbing component may be used in a plumbing component. In some cases, modular wall component 200 includes one or more standard manifolds for the PEX plumbing to minimize connection points where failures are more likely to occur.

In some cases, modular wall component 200 includes a stub-out. In some cases, the stub-out is standard or is pre-manufactured. In some cases, a standard brass stub-out is assembled and coiled/placed inside modular wall component 200 prior to final assembly of modular wall component 200. In some cases, the stub-out is mounted with one or more screws in pre-drilled locations of modular wall component 200 during final assembly of modular wall component 200. In some cases, modular wall component 200 includes PEX plumbing, and the stub-out is at least partially retracted into modular wall component 200 for shipping.

According to some aspects, the plumbing component is manufactured separately from modular wall component 200 and installed after manufacturing. In some cases, the plumbing component is installed in one or more features of the plurality of features 240 of modular wall component 200. According to some aspects, a backing piece is attached to modular wall component 200 and constrains a movement of the plumbing component in the third direction.

According to some aspects, modular wall component 200 includes one or more electrical components, such as a wire, an electrical connection, a panel, an outlet, a switch, etc. In some cases, a load center may be located in modular wall component 200 to provide a convenient location for an electrical panel so electrical wiring may route into modular wall component 200. In some cases, one or more features of the plurality of features 240 are implemented as a pilot hole for an electrical component so a worker may avoid performing secondary alignment at an installation site of modular wall component 200.

According to some aspects, one or more features of the plurality of features 240 are located a sufficient distance in the third direction from a piece of drywall adjacent to or joined to modular wall component 200 so the wiring may be placed in modular wall component 200 without additional hardware.

According to some aspects, the electrical component is a cabinet box (for example, for one or more LED lights). In some cases, the cabinet box includes electrical routing. In some cases, a power supply (e.g., a DC or an AC power supply) and a junction box are embedded in modular wall component 200 via one or more features of the plurality of features 240, and a cover of the junction box is used for access to the power supply.

According to some aspects, the electrical component is a light. In some cases, the light is controlled via a wired or a wireless connection. In some cases, one or more switches may be used with a magnetic back such that a switch may be placed in a surface-mounted junction box cover. In some cases, a light junction box includes a plate at a predetermined depth such that a mud ring is omitted.

FIG. 3 shows an example of a back view of the modular wall component of FIG. 2 according to aspects of the present disclosure. Modular wall component 300 is an example of, or includes aspects of, the corresponding element described with reference to FIGS. 1, 2, and 4-15. A repeated description of one or more similar elements is omitted for the sake of brevity.

In one aspect, modular wall component 300 includes first vertical panel 305, first horizontal panel 310, first interior joint 315, first mounting panel 320, third mounting panel 325, fourth mounting panel joint 330, second buttress 335, third buttress 340, split 345, and first plumbing component 350.

First vertical panel 305 is an example of, or includes aspects of, the corresponding element described with reference to FIGS. 1 and 5. First horizontal panel 310 is an example of, or includes aspects of, the corresponding element described with reference to FIGS. 1, 4, and 6-7. First interior joint 315 is an example of, or includes aspects of, the corresponding element described with reference to FIG. 1. First mounting panel 320 is an example of, or includes aspects of, the corresponding element described with reference to FIGS. 1 and 4-5. Third mounting panel 325 is an example of, or includes aspects of, the corresponding element described with reference to FIG. 1. Second buttress 335 and third buttress 340 are examples of, or include aspects of, a buttress as described with reference to FIG. 1 and the corresponding elements described with reference to FIG. 4. Split 345 is an example of, or includes aspects of, the corresponding element described with reference to FIGS. 1 and 4. First plumbing component 350 is an example of, or includes aspects of, the corresponding element described with reference to FIGS. 2, 4, 6, and 8-10.

FIG. 3 shows a side of the modular wall component of FIG. 2 that faces outwardly in an opposite third direction from the side of the outwardly facing side of the modular wall component shown in FIG. 2. As shown in FIG. 3, first vertical panel 305 and first horizontal panel 310 form first interior joint 315 as a slot joint (indicated within dotted lines). As shown by FIG. 1 and FIG. 3, one or more slot joints (including, e.g., first interior joint 315) provides for a vertical panel (e.g., first vertical panel 305) to contact one or more horizontal panels by slotting into the one or more horizontal panels, and for a horizontal panel (e.g., first horizontal panel 310) to contact one or more vertical panels by slotting into the one or more vertical panels.

As shown in FIG. 3, in some cases, a mounting panel joint (e.g., fourth mounting panel joint 330, indicated within dashed lines) may be formed as a slot joint between a mounting panel (e.g., third mounting panel 325) and a vertical panel (e.g., first vertical panel 305).

As shown in FIG. 3, second buttress 335 and third buttress 340 are disposed adjacent to each other on opposing sides of split 345 in modular wall component 300. In some cases, a buttress (including, e.g., each of second buttress 335 and third buttress 340) may be an integrally positioned component that may provide additional structural support for the modular wall component 300. As shown in FIG. 3, second buttress 335 and third buttress 340 are a mirrored shape of one another, but the present disclosure is not limited thereto, and one or more buttresses may be of different shapes from each other.

As shown in FIG. 3, at least some of first plumbing component 350 is disposed in a space between first mounting panel 320 and third mounting panel 325. As shown in FIG. 3, first mounting panel 320 and third mounting panel 325 are disposed on opposite sides of modular wall component 300 in the third direction.

FIG. 4 shows an example of a side view of the modular wall component of FIG. 2 according to aspects of the present disclosure. Modular wall component 400 is an example of, or includes aspects of, the corresponding element described with reference to FIGS. 1-3 and 5-15. A repeated description of one or more similar elements is omitted for the sake of brevity.

FIG. 4 shows a view facing third vertical panel 402 of modular wall component 400 in the second direction. Third vertical panel 402 is an example of, or includes aspects of, the corresponding element described with reference to FIGS. 1 and 6-7.

In one aspect, modular wall component 400 includes first lattice frame 401, second lattice frame 410, split 414, a plurality of features 415, first mounting panel 416, second mounting panel 417, first mounting panel joint 418, fourth mounting panel joint 419, first buttress 420, buttress joint 421, first plumbing component 422, second plumbing component 423, sixth plumbing component 424, and seventh plumbing component 425.

First lattice frame 401 and second lattice frame 410 are examples of, or include aspects of, the corresponding elements described with reference to FIG. 1. Split 414 is an example of, or includes aspects of, the corresponding element described with reference to FIGS. 1 and 3. The plurality of features 415 is an example of, or includes aspects of, the corresponding element described with reference to FIGS. 1, 2, 7, and 8. First mounting panel 416 is an example of, or includes aspects of, the corresponding element described with reference to FIGS. 1, 3, and 5. Second mounting panel 417 is an example of, or includes aspects of, the corresponding elements described with reference to FIGS. 1 and 7. First mounting panel joint 418 (indicated within dashed lines) is an example of, or includes aspects of, the corresponding element described with reference to FIG. 1. Fourth mounting panel joint 419 (indicated within dashed lines) is an example of, or includes aspects of, the corresponding element described with reference to FIGS. 1 and 7.

First buttress 420 and buttress joint 421 (indicated within dashed lines) are examples of, or include aspects of, the corresponding elements described with reference to FIG. 1. First plumbing component 422 is an example of, or includes aspects of, the corresponding element described with reference to FIGS. 2, 3, 6, and 8-10. Second plumbing component 423 is an example of, or includes aspects of, the corresponding element described with reference to FIGS. 2 and 8. Sixth plumbing component 424 is an example of, or includes aspects of, the corresponding element described with reference to FIGS. 2 and 6. Seventh plumbing component 425 is an example of, or includes aspects of, the corresponding element described with reference to FIG. 2.

In one aspect, first lattice frame 401 includes third vertical panel 402, first horizontal panel 403, second horizontal panel 404, third horizontal panel 405, fourth horizontal panel 406, seventh interior joint 407, eighth interior joint 408, and ninth interior joint 409. First horizontal panel 403 is an example of, or includes aspects of, the corresponding element described with reference to FIGS. 1, 3, and 6-7. Second horizontal panel 404 is an example of, or includes aspects of, the corresponding element described with reference to FIGS. 1 and 5. Third horizontal panel 405 is an example of, or includes aspects of, the corresponding element described with reference to FIGS. 1 and 7. Fourth horizontal panel 406 is an example of, or includes aspects of, the corresponding element described with reference to FIGS. 1 and 5. Seventh interior joint 407 (indicated within dashed lines) is an example of, or includes aspects of, the corresponding element described with reference to FIGS. 1 and 6-7. Eighth interior joint 408 (indicated within dashed lines) is an example of, or includes aspects of, the corresponding element described with reference to FIGS. 1 and 7. Ninth interior joint 409 (indicated within dashed lines) is an example of, or includes aspects of, the corresponding element described with reference to FIG. 1.

In one aspect, second lattice frame 410 includes third additional vertical panel 411, first additional horizontal panel 412, and second additional horizontal panel 413. Third additional vertical panel 411, first additional horizontal panel 412, and second additional horizontal panel 413 are examples of, or include aspects of, the corresponding elements described with reference to FIG. 1.

FIG. 5 shows an example of a horizontal perspective cross-sectional view of the modular wall component of FIG. 2 according to aspects of the present disclosure. Modular wall component 500 is an example of, or includes aspects of, the corresponding element described with reference to FIGS. 1-4 and 6-15. A repeated description of one or more similar elements is omitted for the sake of brevity.

In one aspect, modular wall component 500 includes first vertical panel 505, second horizontal panel 510, third interior joint 515, fourth horizontal panel 520, sixth interior joint 525, first mounting panel 530, and second mounting panel joint 535.

First vertical panel 505 is an example of, or includes aspects of, the corresponding element described with reference to FIGS. 1 and 3. Second horizontal panel 510 is an example of, or includes aspects of, the corresponding element described with reference to FIGS. 1 and 4. Third interior joint 515 (indicated within dashed lines) is an example of, or includes aspects of, the corresponding element described with reference to FIG. 1. Fourth horizontal panel 520 is an example of an outermost horizontal panel described with reference to FIGS. 1 and 4. Sixth interior joint 525 (indicated within dashed lines) is an example of, or includes aspects of, the corresponding element described with reference to FIG. 1. First mounting panel 530 is an example of, or includes aspects of, the corresponding element described with reference to FIGS. 1 and 3. Second mounting panel joint 535 (indicated within dashed lines) is an example of, or includes aspects of, the corresponding element described with reference to FIG. 1.

Referring to FIG. 5, a cross-section of first vertical panel 505 shows the arrangement of respective portions of first vertical panel 505 and second horizontal panel 510 to form third interior joint 515. For example, FIG. 5 shows a cross-section of third interior joint 515, and that third interior joint 515 is a slot joint. Likewise, FIG. 5 shows the arrangement of respective portions of first vertical panel 505 and fourth horizontal panel 520 to form sixth interior joint 525. For example, FIG. 5 shows a cross-section of sixth interior joint 525. Furthermore, FIG. 5 shoes the arrangement of first vertical panel 505 and first mounting panel 530 to form second mounting panel joint 535. For example, FIG. 5 shows a cross-section of second mounting panel joint 535.

FIG. 6 shows an example of a vertical cross-sectional view of the modular wall component of FIG. 2 according to aspects of the present disclosure. Modular wall component 600 is an example of, or includes aspects of, the corresponding element described with reference to FIGS. 1-5 and 7-15. A repeated description of one or more similar elements is omitted for the sake of brevity.

In one aspect, modular wall component 600 includes first horizontal panel 605, second vertical panel 610, second interior joint 615, third vertical panel 620, seventh interior joint 625, first plumbing component 630, fifth plumbing component 635, and sixth plumbing component 640.

First horizontal panel 605 is an example of, or includes aspects of, the corresponding element described with reference to FIGS. 1, 3-4, and 7. Second vertical panel 610 is an example of, or includes aspects of, the corresponding element described with reference to FIGS. 1 and 7. Second interior joint 615 (indicated within dashed lines) is an example of, or includes aspects of, the corresponding element described with reference to FIGS. 1 and 7. Third vertical panel 620 is an example of, or includes aspects of, the corresponding element described with reference to FIGS. 1, 4, and 7. Seventh interior joint 625 (indicated within dashed lines) is an example of, or includes aspects of, the corresponding element described with reference to FIGS. 1, 4, and 7.

First plumbing component 630 is an example of, or includes aspects of, the corresponding element described with reference to FIGS. 2-4 and 8-10. Fifth plumbing component 635 is an example of, or includes aspects of, the corresponding element described with reference to FIGS. 2 and 4. Sixth plumbing component 640 is an example of, or includes aspects of, the corresponding element described with reference to FIG. 2.

Referring to FIG. 6, a cross-section of first horizontal panel 605 shows a top-down view in the first direction of a cross-section of second interior joint 615 formed by first horizontal panel 605 and second vertical panel 610, a cross-section of seventh interior joint 625 formed by first horizontal panel 605 and third vertical panel 620, and a cross-section of first plumbing component 630.

FIG. 7 shows an example of a vertical perspective cross-sectional view of the modular wall component of FIG. 2 according to aspects of the present disclosure. Modular wall component 700 is an example of, or includes aspects of, the corresponding element described with reference to FIGS. 1-6 and 8-15. A repeated description of one or more similar elements is omitted for the sake of brevity.

In one aspect, modular wall component 700 includes first horizontal panel 705, second vertical panel 710, second interior joint 715, third vertical panel 720, seventh interior joint 725, third horizontal panel 730, eighth interior joint 735, second mounting panel 740, third mounting panel joint 745, fourth mounting panel joint 750, and a plurality of features 755.

First horizontal panel 705 is an example of, or includes aspects of, the corresponding element described with reference to FIGS. 1, 3-4, and 6. Third horizontal panel 730 is an example of, or includes aspects of, the corresponding element described with reference to FIGS. 1 and 4. Second vertical panel 710 is an example of, or includes aspects of, the corresponding element described with reference to FIG. 1. Second interior joint 715 (indicated within dashed lines) is an example of, or includes aspects of, the corresponding element described with reference to FIGS. 1 and 6. Third vertical panel 720 is an example of, or includes aspects of, the corresponding element described with reference to FIGS. 1, 4, and 6. Seventh interior joint 725 (indicated within dashed lines) is an example of, or includes aspects of, the corresponding element described with reference to FIGS. 1, 4, and 6. Eighth interior joint 735 (indicated within dashed lines) is an example of, or includes aspects of, the corresponding element described with reference to FIGS. 1 and 4.

Second mounting panel 740 is an example of, or includes aspects of, the corresponding element described with reference to FIGS. 1 and 4. Third mounting panel joint 745 (indicated within dashed lines) and fourth mounting panel joint 750 (indicated within dashed lines) are examples of, or include aspects of, the corresponding elements described with reference to FIGS. 1 and 4. The plurality of features 755 are examples of, or include aspects of, the corresponding element described with reference to FIGS. 1-2 and 8-9.

Referring to FIG. 7, a cross-section of first horizontal panel 705 shows a cross-section of second interior joint 715 formed by first horizontal panel 705 and second vertical panel 710 and a cross-section of seventh interior joint 725 formed by first horizontal panel 705 and third vertical panel 720.

FIG. 7 also shows that eighth interior joint 735 is formed by third vertical panel 720 and third horizontal panel 730, third mounting panel joint 745 is formed by second vertical panel 710 and second mounting panel 740, and fourth mounting panel joint 750 is formed by third vertical panel 720 and second mounting panel 740.

FIG. 7 also shows that a mounting panel (such as second mounting panel 740) may include a plurality of features (such as plurality of features 755). As shown in FIG. 7, the plurality of features may include a series of guide holes for mounting one or more external components to a mounting panel.

FIG. 8 shows an example of a perspective detail view of the modular wall component of FIG. 2 according to aspects of the present disclosure. Modular wall component 800 is an example of, or includes aspects of, the corresponding element described with reference to FIGS. 1-7 and 9-15. A repeated description of one or more similar elements is omitted for the sake of brevity.

In one aspect, modular wall component 800 includes first plumbing component 805, second plumbing component 810, third plumbing component 815, first feature 820, second feature 825, third feature 830, and fourth feature 835. First plumbing component 805 is an example of, or includes aspects of, the corresponding element described with reference to FIGS. 2-4, 7, 9, and 10. Second plumbing component 810 is an example of, or includes aspects of, the corresponding element described with reference to FIGS. 2 and 4. Third plumbing component 815 is an example of, or includes aspects of, the corresponding element described with reference to FIG. 2. Each of first feature 820, second feature 825, third feature 830, and fourth feature 835 is a feature of the plurality of features described with reference to FIGS. 1, 2, 4, 7, and 9.

Referring to FIG. 8, one or more MEP components and/or data infrastructure components may be routed through one or more features. For example, as shown in FIG. 8, first plumbing component 805 is routed through first feature 820, each of second plumbing component 810 and third plumbing component 815 is routed through second feature 825, second plumbing component 810 is routed through third feature 830, and third plumbing component 815 is routed through fourth feature 835.

FIG. 9 shows an example of a perspective detail view of the modular wall component of FIG. 2 according to aspects of the present disclosure. Modular wall component 900 is an example of, or includes aspects of, the corresponding element described with reference to FIGS. 1-8, and 10-15. A repeated description of one or more similar elements is omitted for the sake of brevity.

In one aspect, modular wall component 900 includes first additional vertical panel 905, second additional vertical panel 910, fifth feature 915, sixth feature 920, and first plumbing component 925. First additional vertical panel 905 and second additional vertical panel 910 are examples of, or include aspects of, the corresponding elements described with reference to FIG. 1. In one aspect, each of fifth feature 915 and sixth feature 920 are features of the plurality of features described with reference to FIGS. 1, 2, 4, 7, and 8. First plumbing component 925 is an example of, or includes aspects of, the corresponding element described with reference to FIGS. 2-4, 7, 8, and 10.

Referring to FIG. 9, in some cases, one or more features are oriented to provide a natural slope to a component at least partially disposed within the one or more features. For example, in some cases, a non-pressurized plumbing component may operate using gravity, and so may be pitched at a downwardly sloping angle. As shown in FIG. 9, a bottom of fifth feature 915 is disposed at a higher height in the first direction than a bottom of sixth feature 920, providing an ability for first plumbing component 925 to respectively pass through the first additional vertical panel 905 and second additional vertical panel 910 at a downwardly sloping angle so that a fluid may pass through first plumbing component 925 via gravity.

In some cases, one or more features of a modular wall component (such as modular wall component 900) are oversized for simple installation and adjustment of a component during or after installation of the modular wall component. For example, in some cases, prior to shipment of the modular wall component to an installation site, a plumbing component, a mechanical component, an electric component, or a combination thereof may be temporarily set within the modular wall component. In some cases, when the modular wall component is ready to be installed, a user can unset the temporary sets and permanently or semi-permanently set the plumbing component, the mechanical component, the electric component, or the combination thereof in place.

FIG. 10 shows an example of a front detail view of the modular wall component of FIG. 2 according to aspects of the present disclosure. Modular wall component 1000 is an example of, or includes aspects of, the corresponding element described with reference to FIGS. 1-9 and 11-15. A repeated description of one or more similar elements is omitted for the sake of brevity.

In one aspect, modular wall component 1000 includes first plumbing component 1005, fourth mounting panel 1010, and fifth mounting panel 1015.

First plumbing component 1005 is an example of, or includes aspects of, the corresponding element described with reference to FIGS. 2-4 and 7-9. Fourth mounting panel 1010 and fifth mounting panel 1015 are examples of, or include aspects of, the corresponding elements described with reference to FIG. 1.

Referring to FIG. 10, fourth mounting panel 1010 and fifth mounting panel 1015 are disposed on opposing sides of modular wall component 1000 in the third direction, and first plumbing component 1005 is disposed in a space between fourth mounting panel 1010 and fifth mounting panel 1015.

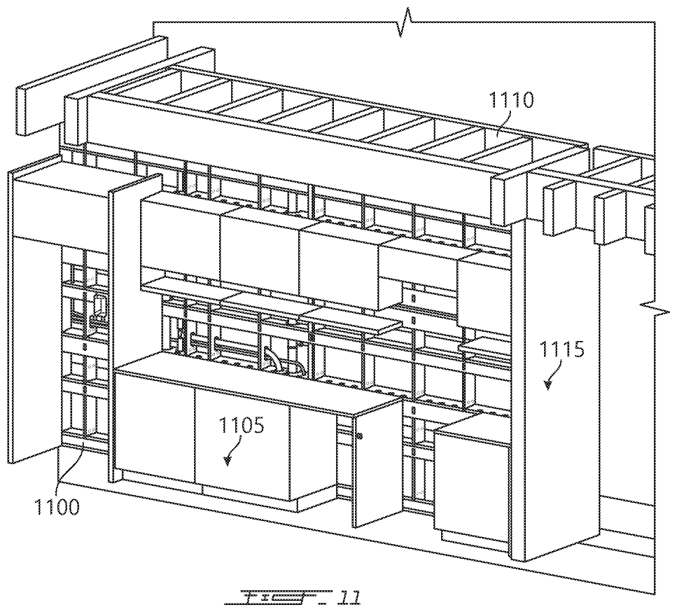

FIG. 11 shows an example of a modular wall component 1100 installation according to aspects of the present disclosure. The example shown includes modular wall component 1100, first external structure 1105, second external structure 1110, and third external structure 1115. Modular wall component 1100 is an example of, or includes aspects of, the corresponding element described with reference to FIGS. 1-10, and 12-15. A repeated description of one or more similar elements is omitted for the sake of brevity.

Referring to FIG. 11, in some cases, a modular wall component such as modular wall component 1100 is installed alongside one or more external structures. As shown, modular wall component 1100 is installed alongside first external structure 1105 (e.g., a set of cabinets), second external structure 1110 (e.g., a ceiling structure), and third external structure 1115 (e.g., a wall structure). For example, in some cases, FIG. 11 shows a kitchen. In some cases, each of first external structure 1105, second external structure 1110, and third external structure 1115 are joined with modular wall component 1100 at various points of modular wall component 1100 (including, for example, a mounting panel, a vertical panel, a horizontal panel, or a combination thereof as described with reference to FIG. 1). In some cases, a reinforcement component (such as a brace, a panel, a bar, etc.) is included in modular wall component 1100 to help modular wall component 1100 to support at least one of first external structure 1105, second external structure 1110, or third external structure 1115.

In some cases, one or more cabinets of first external structure 1105 may be expanded or retracted to be set to different depths away from modular wall component 1100. For example, if a customer desires a thicker backsplash in the kitchen, a user may move a cabinet box in relation to modular wall component 1100 without using a secondary extension piece. In some cases, a degree of recession of one or more outlets (or other components) into modular wall component 1100 can be likewise adjusted.

As shown, modular wall component 1100 can include a plurality of lattice frames as described with reference to FIG. 1. In some cases, a finish panel (such as a drywall panel) is disposed between modular wall component 1100 and first external structure 1105. A finish panel is omitted from FIG. 11 between modular wall component 1100 and first external structure 1105 for an ease of illustrating modular wall component 1100.

According to some aspects, a modular wall component such as modular wall component 1100 may act as a structural substrate, replacing a traditionally framed wall. For example, the traditionally framed wall may be an array of vertical or horizontal sections of lumber, extending from a floor to a ceiling structure. The traditionally framed wall may include lumber that is adjacent to MEP componentry, as well as insulation. For example, in some cases, modular wall component 1100 is disposed between a first and second layer of drywall, between tongue and groove paneling, or between any other suitable external finish material. Accordingly, in some cases, modular wall component 1100 can function as either a structural wall or as a non-structural wall.

In some cases, a modular wall component such as modular wall component 1100 may abut against a traditionally framed wall component (such as third external structure 1115), but embodiments of the present disclosure are not limited thereto. For example, in some cases, modular wall component 1100 may abut against a wall including cross-laminated timber (CLT), mass timber, glue-laminated timber (GLT), a mass plywood panel, or other engineered timber product. In some cases, modular wall component 1100 abuts against drywall, tongue and groove paneling, or any other suitable external finish material. According to some aspects, shelving and finish materials can be installed in or on predetermined locations of modular wall component 1100 for a seamless appearance.

According to some aspects, one or more modular wall components 1100 may be used in any orientation or configuration to provide a building interior configuration. For example, in some cases, a kitchen may be adjacent to a laundry room. As a result, one or more modular wall components 1100 may be disposed within and/or between the kitchen and the laundry room to provide MEP componentry for the kitchen and the laundry room.

### Installing a Modular Wall Component

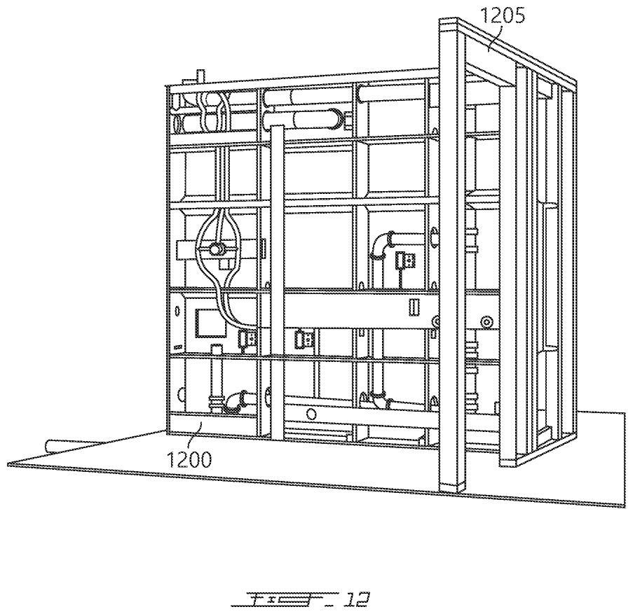

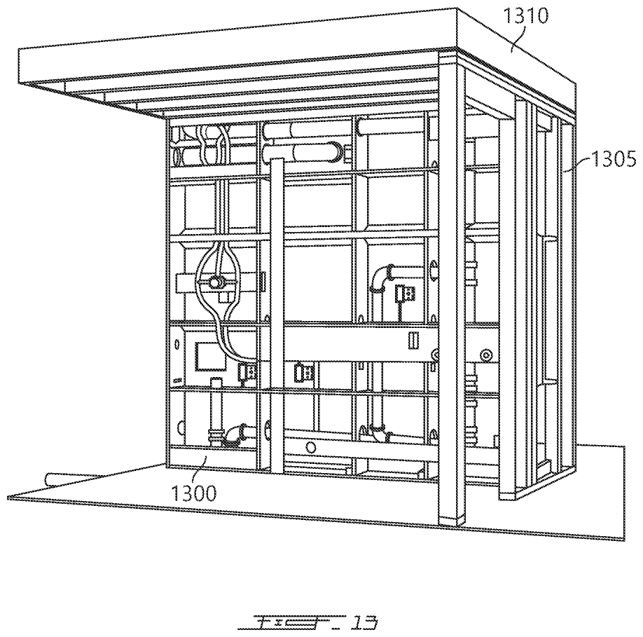

FIGS. 12-15 show an example of installing a modular wall component according to aspects of the present disclosure. The example shown in FIG. 12 includes modular wall component 1200 and adjacent wall 1205. Modular wall component 1200 is an example of, or includes aspects of, the corresponding element described with reference to FIGS. 1-11, and 13-15. Adjacent wall 1205 is an example of, or includes aspects of, the corresponding element described with reference to FIGS. 13-15.

Referring to FIG. 12, in an example, modular wall component 1200 is installed at an installation site (for example, on a floor). In some cases, modular wall component 1200 is adjusted prior to or during installation (for example, by adjusting a position, size, or orientation of one or more elements of modular wall component 1200) to accommodate a tolerance of the floor. Adjacent wall 1205 is joined to modular wall component 1200 (for example, by a joint, a screw, a bolt, a bracket, etc.).

In some cases, a lower piece of modular wall component 1200 (such as a lower lattice frame) includes a cutout so modular wall component 1200 can be placed where, for example, a cabinet would be placed. Then, modular wall component 1200 can be slid into place without touching or affecting, for example, a plumbing component or an electrical component that may be disposed outside of surrounding material (such as concrete). Then, the upper piece of the two pieces (e.g., an upper lattice frame) can be set on the lower piece, separated from an exterior or rearward wall, and slid into place. As a result, connections for an external appliance, such as a microwave, a solar conduit, etc., can be made prior to full installation of modular wall component 1200. Therefore, a simple and easy process for installation of modular wall component 1200 may be achieved.

In some cases, a split in modular wall component 1200 (such as the split described with reference to FIG. 1) may be oriented based on one or more sink stub outs. For instance, in some cases, an area around one or more sink stub outs may be empty and may include a connection to a foundation of the installation site. In some cases, by moving the split to the one or more sink stub outs, more electrical componentry may be included in the load center, such as the microwave or water heater. Additionally or alternatively, in some cases, water lines are run further up in modular wall component 1200 towards the water heater.

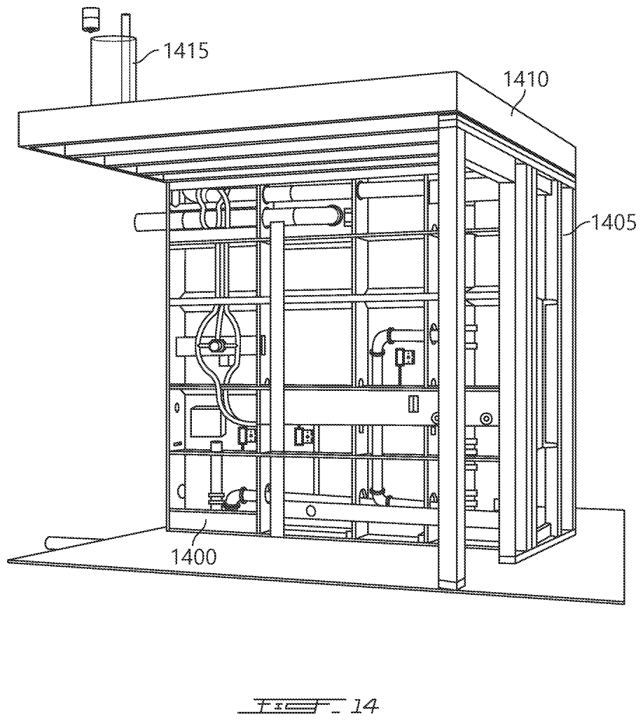

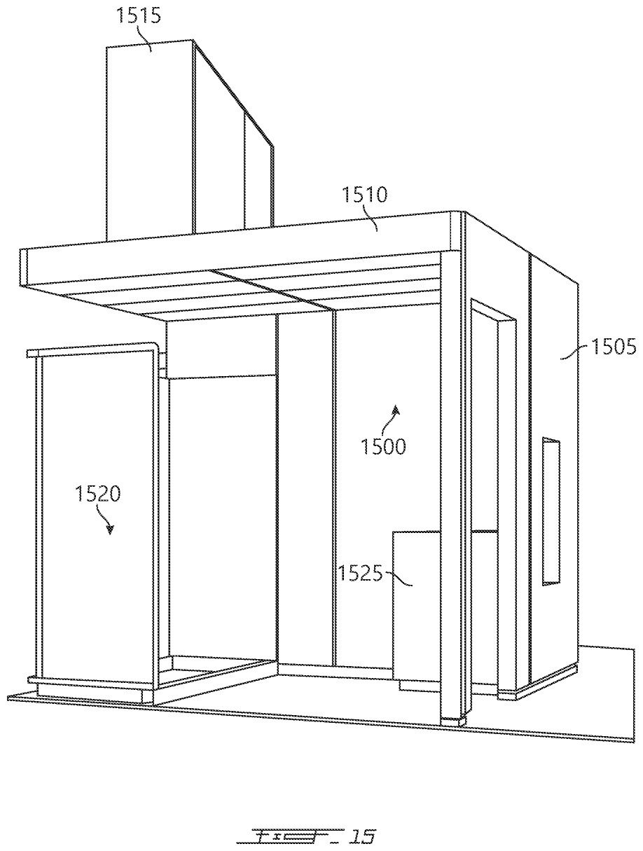

The example shown in FIG. 13 includes modular wall component 1300, adjacent wall 1305, and adjacent ceiling 1310. Modular wall component 1300 is an example of, or includes aspects of, the corresponding element described with reference to FIGS. 1-12, 14, and 15. Adjacent wall 1305 is an example of, or includes aspects of, the corresponding element described with reference to FIGS. 12 and 14-15. Adjacent ceiling 1310 is an example of, or includes aspects of, the corresponding element described with reference to FIGS. 14-15.

Referring to FIG. 13, adjacent ceiling 1310 is attached to modular wall component 1300 and adjacent wall 1305 (for example, by a joint, a screw, a bolt, a bracket, etc.). In some cases, modular wall component 1300 is adjusted prior to or during installation (for example, by adjusting a position, size, or orientation of one or more elements of modular wall component 1200) to accommodate a tolerance of adjacent ceiling 1310.

The example shown in FIG. 14 includes modular wall component 1400, adjacent wall 1405, adjacent ceiling 1410, and additional componentry 1415. Modular wall component 1400 is an example of, or includes aspects of, the corresponding element described with reference to FIGS. 1-13, and 15. Adjacent wall 1405 is an example of, or includes aspects of, the corresponding element described with reference to FIGS. 12-13 and 15. Adjacent ceiling 1410 is an example of, or includes aspects of, the corresponding element described with reference to FIGS. 13 and 15. Additional componentry 1415 is an example of, or includes aspects of, the corresponding element described with reference to FIG. 15.

Referring to FIG. 14, additional componentry 1415 (such as a water heater) is installed on adjacent ceiling 1410. In some cases, additional componentry 1415 is connected to at least one of a mechanical component, an electrical component, or a plumbing component disposed in modular wall component 1400.

In an example, armored cables may be used for the water heater. In some cases, the armored cable is coiled up at the top of modular wall component 1400 such that the armored cable runs straight to the water heater without additional paneling added to modular wall component 1400. In another example, a conduit line disposed in modular wall component 1400 may be used to attach to additional componentry 1415 (such as a solar panel). The conduit line may be disposed such that the conduit line can be tied straight into a load center punch out, high enough that an elbow could be fit, and routed through an exterior wall via a flexible gasket seal.

The example shown in FIG. 15 includes modular wall component 1500, adjacent wall 1505, adjacent ceiling 1510, additional componentry 1515, shower 1520, and sink 1525. Modular wall component 1500 is an example of, or includes aspects of, the corresponding element described with reference to FIGS. 1-14. Adjacent wall 1505 is an example of, or includes aspects of, the corresponding element described with reference to FIGS. 12-14. Adjacent ceiling 1510 is an example of, or includes aspects of, the corresponding element described with reference to FIGS. 13-14. Additional componentry 1515 is an example of, or includes aspects of, the corresponding element described with reference to FIG. 14.

Referring to FIG. 15, finishing surfaces/components can be applied to one or more of modular wall component 1500, adjacent wall 1505, adjacent ceiling 1510, and additional componentry 1515. For example, as shown in FIG. 15, finishing surfaces are applied to modular wall component 1500, adjacent wall 1505, adjacent ceiling 1510, additional componentry 1515 is enclosed in a cabinet, and shower 1520 and sink 1525 are installed and connected to one or more components of modular wall component 1500 and additional componentry 1515 to supply plumbing service to shower 1520 and sink 1525.

In some implementations, modular wall component 1500 can include a finish panel for an aesthetically pleasing appearance for an end-user. In an example, the finish panel may include one or more strips of plywood. In some cases, the seams of the plywood (e.g., where the strips of plywood abut against one another) may be located to be hidden by shelves and cabinets such that the end-user cannot see fasteners for attaching the plywood to modular wall component 1200. For example, the plywood strips may be located at a height from a specified lower datum to provide a solid structure for second finishing material to be mounted, such as drywall. As a result, in some cases, shelving and cabinets mounted to modular wall component 1500 provide a clean appearance. Additionally or alternatively, a small undercut may be used such that a bottom edge of a panel of modular wall component 1500 is reduced or eliminated during an alignment of a lower piece of modular wall component 1500 to the floor and an upper piece of modular wall component 1500 to lower piece.

Panels may be set in such a way to mount a shower door and sides to a shower. Additionally, the panels around the shower may be pre-manufactured with cutouts for stub outs and temperature control units to be installed.

### Manufacturing a Modular Wall Component

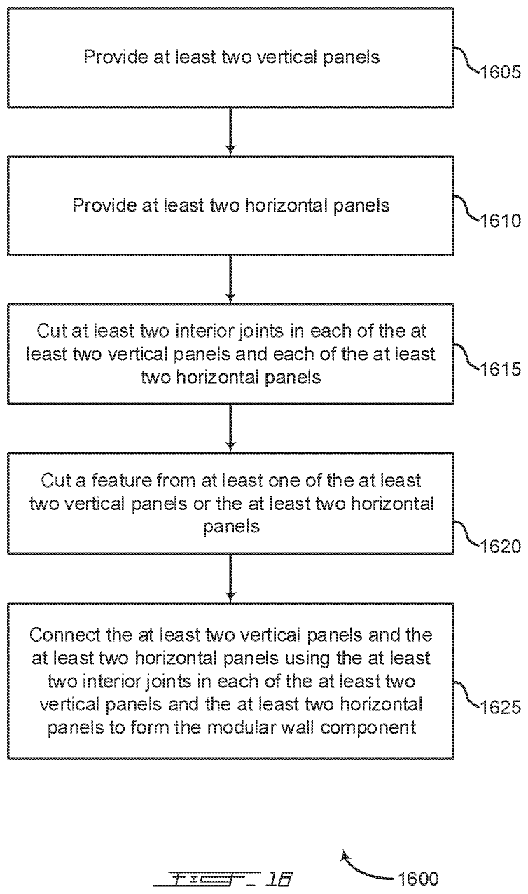

FIG. 16 shows an example of a method 1600 for providing a modular wall component according to aspects of the present disclosure. In some examples, the operations of FIG. 16 may be performed by a system including a processor executing a set of codes to control functional elements of an apparatus. Additionally or alternatively, certain processes are performed using special-purpose hardware. Generally, the operations of FIG. 16 are performed according to the methods and processes described in accordance with aspects of the present disclosure. In some cases, the operations described herein are composed of various substeps, or are performed in conjunction with other operations.

Referring to FIG. 16, in some cases, the system (such as a computer system including one or more processors executing instructions stored in a memory) provides components that can be assembled to form a modular wall component (such as a modular wall component described with reference to FIGS. 1-15).

For example, in some cases, a user measures an installation site for the modular wall component and determines a location for the modular wall component at the installation site. In some cases, the user determines dimensions for the modular wall component based on the measurement of the installation site including a height in the first direction, a width in the second direction, and a depth in the third direction described with reference to FIG. 1.

In some cases, based on the dimensions for the installation site, the user designs dimensions for a vertical panel and a horizontal panel as described with reference to FIG. 1 (for example, using computer-aided design (CAD) software provided by the system on a user device). In some cases, the user designs an interior joint for the vertical panel and the horizontal panel (for example, using the CAD software). In some cases, the user designs a location, shape, size, and orientation for at least one feature to be included in the vertical panel or the horizontal panel (for example, using the CAD software). In some cases, the user designs the feature based on a mechanical component, an electrical component, a plumbing component, or a combination thereof to be included in the modular wall component (for example, using the CAD software). In some cases, the user designs a mounting panel to be included in the modular wall component (for example, using the CAD software).

In some cases, the user creates a numerical control (NC) program based on the design of the vertical panel, the horizontal panel, and the feature (for example, using the system). In some cases, the user creates the NC program based on the design of the mounting panel. In some cases, the user provides the NC program to a computer numerical control (CNC) machine included in the system.

At operation 1605, the system provides at least two vertical panels. For example, in some cases, the CNC machine cuts the at least two vertical panels from sheet stock wood according to the NC program.

At operation 1610, the system provides at least two horizontal panels. For example, in some cases, the CNC machine cuts the at least two horizontal panels from sheet stock wood according to the NC program.

In some cases, the system provides a mounting panel. For example, in some cases, the CNC machine cuts the mounting panel from sheet stock wood according to a mounting panel design included in the NC program.

At operation 1615, the system cuts at least two interior joints (or respective portions of the at least two interior joints) in each of the at least two vertical panels and each of the at least two horizontal panels. For example, in some cases, the CNC machine cuts portions from the at least two vertical panels and the at least two horizontal panels to form the at least two interior joints (or the respective portions of the at least two interior joints) according to the NC program.

In some cases, the system cuts a mounting panel joint (or a respective portion of a mounting panel joint) from the vertical panel (or the horizontal panel) and the mounting panel using the CNC machine according to a mounting panel joint design included in the NC program.

At operation 1620, the system cuts a feature from at least one of the at least two vertical panels or the at least two horizontal panels. For example, in some cases, the CNC machine cuts the feature according to the NC program. In some cases, the CNC machine cuts the feature from the mounting panel.

At operation 1625, the at least two vertical panels and the at least two horizontal panels are connected using the at least two interior joints in each of the at least two vertical panels and the at least two horizontal panels to form the modular wall component. For example, in some cases, a user or an assembly machine (such as a robot) operated by or included in the system joins the at least two vertical panels and the at least two horizontal panels using the at least two interior joints to form the modular wall component. In some cases, each edge of the at least two horizontal panels forms an edge of a set of internal rectangular spaces defined by the at least two vertical panels. In some cases, the user or the assembly machine attaches the mounting panel to the modular wall component via the mounting panel joint.

In some cases, a user or an assembly machine (such as a robot) operated by or included in the system installs at least one of a mechanical component, an electrical component, and a plumbing component in the modular wall component such the mechanical component, the electrical component, and/or the plumbing component is disposed in the feature.

In some cases, the modular wall component is provided to the installation site. In some cases, the modular wall component is installed at the installation site by a user or an assembly machine (such as a robot) operated by or included in the system. In some cases, a user or an assembly machine (such as a robot) operated by or included in the system connects one or more external components to the modular wall component or an element of the modular wall component. In some cases, a user or an assembly machine (such as a robot) operated by or included in the system installs a finish material (e.g., drywall, tongue and groove paneling, etc.) on the modular wall component.

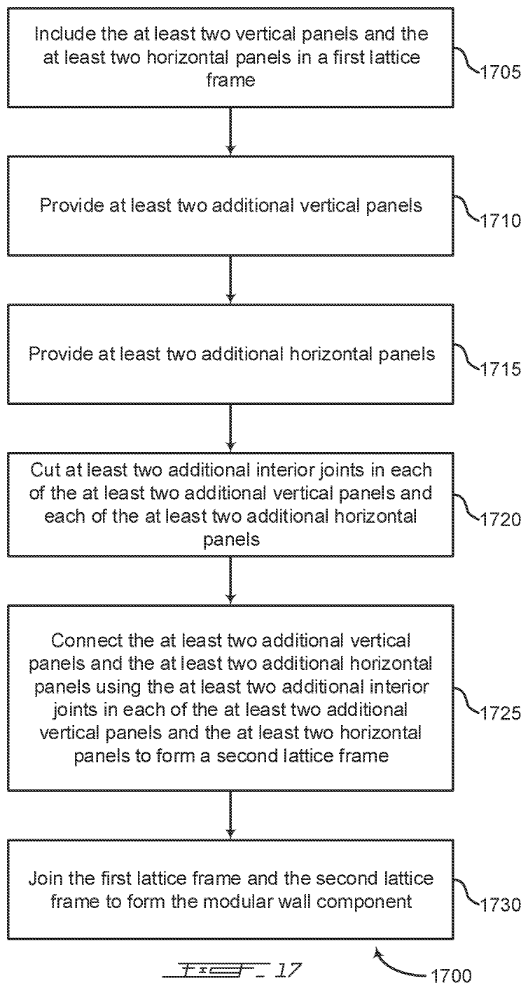

A method for providing a modular wall component including the second lattice frame is described with reference to FIG. 17.

FIG. 17 shows an example of a method 1700 for providing a modular wall component including the second lattice frame according to aspects of the present disclosure. In some examples, the operations of FIG. 17 are performed by a system including a processor executing a set of codes to control functional elements of an apparatus. Additionally or alternatively, certain processes are performed using special-purpose hardware. Generally, the operations of FIG. 17 are performed according to the methods and processes described in accordance with aspects of the present disclosure. In some cases, the operations described herein are composed of various substeps, or are performed in conjunction with other operations.

Referring to FIG. 17, in some cases, the system (such as a computer system including one or more processors executing instructions stored in a memory) provides components that can be assembled to form a modular wall component (such as a modular wall component described with reference to FIGS. 1-15) including two or more lattice frames.

For example, in some cases, a user measures an installation site for the modular wall component and determines a location for the modular wall component at the installation site. In some cases, the user determines dimensions for the modular wall component based on the measurement of the installation site including a height in the first direction, a width in the second direction, and a depth in the third direction described with reference to FIG. 1.

In some cases, based on the dimensions for the modular wall component, the user designs dimensions for a vertical panel and a horizontal panel for a first lattice frame and an additional vertical panel and an additional horizontal panel for a second lattice frame as described with reference to FIG. 1 (for example, using computer-aided design (CAD) software provided by the system on a user device). In some cases, the user designs an interior joint for the vertical panel and the horizontal panel (for example, using the CAD software) and an additional interior joint for the additional vertical panel and the additional horizontal panel (for example, using the CAD software).

In some cases, the user designs the dimensions for the first lattice frame and second lattice frame according to a split in the modular wall component as described with reference to FIG. 1. In some cases, the split determines the design of the vertical panel, the horizontal panel, the additional vertical panel, and the additional horizontal panel. In some cases, the location and/or the orientation of the split in the modular wall component is determined based on: a weight of the first lattice frame and/or the second lattice frame; a location and/or number of external components to be installed along with or attached to the modular wall component at the installation site; a geometry of the installation site such that each of the first lattice frame and the second lattice frame can be effectively and efficiently conveyed to the installation site (for example, so that the first lattice frame and the second lattice frame can be conveyed through a staircase, a doorway, a corner, etc. leading to the installation site); or a combination thereof.

In some cases, the user designs a location, shape, size, and orientation for at least one feature to be included in the vertical panel, the horizontal panel, the additional vertical panel, or the additional horizontal panel (for example, using the CAD software). In some cases, the user designs the feature based on a mechanical component, an electrical component, a plumbing component, or a combination thereof to be included in the modular wall component (for example, using the CAD software).

In some cases, the user creates a numerical control (NC) program based on the design of the first lattice frame and the second lattice frame. In some cases, the user provides the NC program to a computer numerical control (CNC) machine included in the system.

At operation 1705, the at least two vertical panels and the at least two horizontal panels are included in a lattice frame. For example, in some cases, the modular wall component formed by the system at operation 1625 of FIG. 16 is implemented as the lattice frame.

At operation 1710, the system provides at least two additional vertical panels. For example, in some cases, the CNC machine cuts the at least two additional vertical panels from sheet stock wood according to the NC program.

At operation 1715, the system provides at least two additional horizontal panels. For example, in some cases, the CNC machine cuts the at least two additional horizontal panels from sheet stock wood according to the NC program.

At operation 1720, the system cuts at least two additional interior joints (or respective portions of the at least two interior joints) in each of the at least two additional vertical panels and each of the at least two additional horizontal panels. For example, in some cases, the CNC machine cuts portions from the at least two additional vertical panels and the at least two additional horizontal panels to form the at least two additional interior joints (or the respective portions of the at least two additional interior joints) according to the NC program.

At operation 1725, the at least two additional vertical panels and the at least two additional horizontal panels are connected using the at least two additional interior joints in each of the at least two additional vertical panels and the at least two horizontal panels to form a second lattice frame. For example, in some cases, a user or an assembly machine (such as a robot) operated by or included in the system assembles joins the at least two additional vertical panels and the at least two additional horizontal panels using the at least two additional interior joints to form the second lattice frame. In some cases, each edge of the at least two additional horizontal panels forms an edge of a set of additional internal rectangular spaces defined by the at least two additional vertical panels. In some cases, the user or the assembly machine attaches a mounting panel to the second lattice frame via a mounting panel joint provided by the CNC machine.

At operation 1730, the first lattice frame and the second lattice frame are joined to form the modular wall component. For example, in some cases, a user or an assembly machine (such as a robot) operated by or included in the system joins the first lattice frame to the second lattice frame using a joint cut into the first lattice frame and the second lattice frame, a bracket, a bolt, a screw, a magnet, adhesive, friction, or other attachment mechanism. In some cases, the first lattice frame and the second lattice frame are joined to form the modular wall component at the installation site.

Accordingly, the present disclosure includes the following aspects.

A modular wall component is described. One or more aspects of the modular wall component include at least two vertical panels; at least two horizontal panels configured to form a lattice frame together with the at least two vertical panels, where each of the at least two horizontal panels is connected to each of the at least two vertical panels by an interior joint, respectively; and a feature configured to secure a mechanical component, an electrical component, or a plumbing component. In some aspects, the interior joint comprises a slot joint.

In some aspects, at least one vertical panel of the at least two vertical panels or at least one horizontal panel of the at least two horizontal panels comprises a wood panel. In some aspects, the feature comprises a slot, where a portion of the mechanical component, the electrical component, or the plumbing component is disposed in the slot.

Some examples of the modular wall component include a mounting panel joined to an external face of at least one of the at least two vertical panels by a slot joint. Some examples of the modular wall component further include an external structure mounted to the mounting panel at a load attachment point. Some examples of the modular wall component further include a reinforcement component configured to reinforce the load attachment point.

Some examples of the modular wall component further include a second lattice frame joined to the lattice frame and comprising at least two additional vertical panels and at least two additional horizontal panels, where each of the at least two additional horizontal panels is connected to each of the at least two additional vertical panels by an additional interior joint, respectively. In some cases, a split between the lattice frame and the second lattice frame is oriented based on a geometry of an external structure.

In some cases, a split between the lattice frame and the second lattice frame is oriented based on a geometry of the at least one of the mechanical component, the electrical component, or the plumbing component. In some cases, a split between the lattice frame and the second lattice frame is determined based on a weight of the lattice frame or a weight of the second lattice frame.

Some examples of the modular wall component further include a plurality of features configured to accommodate an alignment of the mechanical component, the electrical component, or the plumbing component.

A modular wall component is described. One or more aspects of the modular wall component include at least two vertical panels; at least two horizontal panels configured to form a lattice frame together with the at least two vertical panels, where each of the at least two horizontal panels forms an edge of a plurality of internal rectangular spaces defined by the at least two vertical panels; and a feature configured to secure a mechanical component, an electrical component, or a plumbing component.

Some examples of the modular wall component further include a mounting panel joined to an external face of at least one of the at least two vertical panels. In some aspects, the mounting panel is joined to at least one of the at least two horizontal panels. In some aspects, the mounting panel is at least partially overlapped by at least one vertical panel or at least one horizontal panel.

A method for manufacturing a modular wall component is described. One or more aspects of the method include providing at least two vertical panels; providing at least two horizontal panels; cutting at least two interior joints in each of the at least two vertical panels and each of the at least two horizontal panels; cutting a feature from the at least two vertical panels or the at least two horizontal panels; and connecting the at least two vertical panels and the at least two horizontal panels using the at least two interior joints in each of the at least two vertical panels and the at least two horizontal panels to form the modular wall component.

Some examples of the method further include including the at least two vertical panels and the at least two horizontal panels in a first lattice frame. Some examples further include providing at least two additional vertical panels. Some examples further include providing at least two additional horizontal panels. Some examples further include cutting at least two additional interior joints in each of the at least two additional vertical panels and each of the at least two additional horizontal panels. Some examples further include connecting the at least two additional vertical panels and the at least two additional horizontal panels using the at least two additional interior joints in each of the at least two additional vertical panels and the at least two horizontal panels to form a second lattice frame. Some examples further include joining the first lattice frame and the second lattice frame to form the modular wall component.

Some examples of the method further include determining a split between the lattice frame and the second lattice frame, where the at least two vertical panels, the at least two horizontal panels, the at least two additional vertical panels, the at least two additional horizontal panels, or a combination thereof are provided based on the split.

Some examples of the method further include determining the split based on a mechanical component, an electrical component, a plumbing component, an appliance, a geometry of an external structure, a dimension of the lattice frame, a dimension of the second lattice frame, or a combination thereof.

The description and drawings described herein represent example configurations and do not represent all the implementations within the scope of the claims. For example, the operations and steps may be rearranged, combined, or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein, but is to be accorded the broadest scope consistent with the principles and novel features disclosed herein.

The described methods may be implemented or performed by devices that include a general-purpose processor, a digital signal processor (DSP), an application specific integrated circuit (ASIC), a field programmable gate array (FPGA) or other programmable logic device, discrete gate or transistor logic, discrete hardware components, or any combination thereof. A general-purpose processor may be a microprocessor, a conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). Thus, the functions described herein may be implemented in hardware or software and may be executed by a processor, firmware, or any combination thereof. If implemented in software executed by a processor, the functions may be stored in the form of instructions or code on a computer-readable medium.

Computer-readable media includes both non-transitory computer storage media and communication media including any medium that facilitates transfer of code or data. A non-transitory storage medium may be any available medium that can be accessed by a computer. For example, non-transitory computer-readable media can comprise random access memory (RAM), read-only memory (ROM), electrically erasable programmable read-only memory (EEPROM), compact disk (CD) or other optical disk storage, magnetic disk storage, or any other non-transitory medium for carrying or storing data or code.

Also, connecting components may be properly termed computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

What is claimed is:

1. A modular wall component, comprising:
at least two vertical panels;
at least two horizontal panels configured to form a lattice frame together with the at least two vertical panels, wherein each of the at least two horizontal panels is connected to each of the at least two vertical panels by an interior joint, respectively; and
a feature configured to secure a mechanical component, an electrical component, or a plumbing component.

2. The modular wall component of claim 1, wherein:
at least one vertical panel of the at least two vertical panels or at least one horizontal panel of the at least two horizontal panels comprises a wood panel.

3. The modular wall component of claim 1, wherein:
the feature comprises a slot, wherein a portion of the mechanical component, the electrical component, or the plumbing component is disposed in the slot.

4. The modular wall component of claim 1, further comprising:
a mounting panel joined to an external face of at least one of the at least two vertical panels by a slot joint.

5. The modular wall component of claim 4, further comprising:
an external structure mounted to the mounting panel at a load attachment point.

6. The modular wall component of claim 5, further comprising:
a reinforcement component configured to reinforce the load attachment point.

7. The modular wall component of claim 1, wherein:
the interior joint comprises a slot joint.

8. The modular wall component of claim 1, further comprising:
a second lattice frame joined to the lattice frame and comprising at least two additional vertical panels and at least two additional horizontal panels, wherein each of the at least two additional horizontal panels is connected to each of the at least two additional vertical panels by an additional interior joint, respectively.

9. The modular wall component of claim 8, wherein:
a split between the lattice frame and the second lattice frame is oriented based on a geometry of an external structure.

10. The modular wall component of claim 8, wherein:
a split between the lattice frame and the second lattice frame is oriented based on a geometry of the mechanical component, the electrical component, or the plumbing component.

11. The modular wall component of claim 8, wherein:
a split between the lattice frame and the second lattice frame is determined based on a weight of the lattice frame or a weight of the second lattice frame.

12. The modular wall component of claim 1, further comprising:
a plurality of features configured to accommodate an alignment of the mechanical component, the electrical component, or the plumbing component.

13. A modular wall component, comprising:
at least two vertical panels;
at least two horizontal panels configured to form a lattice frame together with the at least two vertical panels, wherein each of the at least two horizontal panels forms an edge of a plurality of internal rectangular spaces defined by the at least two vertical panels; and
a feature configured to secure a mechanical component, an electrical component, or a plumbing component.

14. The modular wall component of claim 13, further comprising:
a mounting panel joined to an external face of at least one of the at least two vertical panels.

15. The modular wall component of claim 14, wherein:
the mounting panel is joined to at least one of the at least two horizontal panels.

16. The modular wall component of claim 14, wherein:
the mounting panel is at least partially overlapped by at least one vertical panel or at least one horizontal panel.

17. A method for manufacturing a modular wall component, comprising:
providing at least two vertical panels;
providing at least two horizontal panels;
cutting at least two interior joints in each of the at least two vertical panels and each of the at least two horizontal panels;
cutting a feature from the at least two vertical panels or the at least two horizontal panels; and
connecting the at least two vertical panels and the at least two horizontal panels using the at least two interior joints in each of the at least two vertical panels and the at least two horizontal panels to form the modular wall component.

18. The method of claim 17, further comprising:
including the at least two vertical panels and the at least two horizontal panels in a first lattice frame;
providing at least two additional vertical panels;
providing at least two additional horizontal panels;
cutting at least two additional interior joints in each of the at least two additional vertical panels and each of the at least two additional horizontal panels;
connecting the at least two additional vertical panels and the at least two additional horizontal panels using the at least two additional interior joints in each of the at least two additional vertical panels and the at least two horizontal panels to form a second lattice frame; and
joining the first lattice frame and the second lattice frame to form the modular wall component.

19. The method of claim 18, further comprising:
determining a split between the first lattice frame and the second lattice frame, wherein the at least two vertical panels, the at least two horizontal panels, the at least two additional vertical panels, the at least two additional horizontal panels, or a combination thereof are provided based on the split.

20. The method of claim 19, further comprising:
determining the split based on a mechanical component, an electrical component, a plumbing component, an appliance, a geometry of an external structure, a dimension of the first lattice frame, a dimension of the second lattice frame, or a combination thereof.
